SEQUENCE LISTING (26 sequences)

SEQ ID NO: 1 - DNA, 1269 bp, Homo sapiens
  CDS (1)..(1269)
atgggcatgacacggatgctcctggaatgcagtctcagtgacaagttgtgtgtcatccaggagaagcagtatgaagtgattatcgtcccaactttgttggttactatcttcctcatccttcttggggtcatcctgtggctttttatcagagaacaaagaactcaacagcagcgttctggacctcaaggcattgcccctgttcctccacctagggacctaagctgggaagcaggacatggaggaaatgtggctttgccacttaaggagacatccgtggaaaactttctgggagctaccacacctgccctggctaagctgcaggtgccgcgggagcaactctctgaagttctggagcagatttgcagcggtagctgtgggcccatctttcgagccaatatgaacactggggacccttctaagcccaagagtgttattctcaaggctttaaaagaaccagctgggctccatgaggtacaagatttcttagggcgaatccaattccatcaatacctggggaaacacaaaaacctggtgcagctggaaggctgctgcactgaaaagctgccactctatgtggtgttggaggatgtggcccagggggacctgcttagctttctctggacctgtcggcgggatgtgatgactatggatggtcttctctatgatctcacagaaaaacaagtatatcacatcggaaagcaggtccttttggcgctggaattcctgcaggagaagcatttgttccatggggatgtggcagccaggaatattctgatgcaaagtgatctcactgctaagctctgtggattaggcctggcttatgaagtttacacccgaggggccatctcctctactcaaaccatacctctcaagtggcttgccccagaacggcttctcctgagacctgctcgcatcagagcagatgtctggtcttttgggatcctgctctatgagatggtgactctaggagcaccaccgtatcctgaagtccctcctaccagcatcctagagcatctccaaagaaggaaaatcatgaagagacccagtagctgcacacataccatgtacagtatcatgaagtcctgctggcgctggcgtgaggctgaccgcccctcacctagagagctgcgcttgcgcctagaagctgccattaaaactgcagatgacgaggctgtgttacaagtaccagagttggtggtacctgaactgtatgcagctgtggccggcatcagagtggagagcctcttctacaactatagcatgctttga

SEQ ID NO: 2 - protein, 422 aa, Homo sapiens [sequence omitted]

SEQ ID NO: 3 - DNA, 1296 bp, Homo sapiens
  CDS (1)..(1296)
atgggcatgacacggatgctcctggaatgcagtctcagtgacaagttgtgtgtcatccaggagaagcagtatgaagtgattatcgtcccaactttgttggttactatcttcctcatccttcttggggtcatcctgtggctttttatcagagaacaaagaactcaacagcagcgttctggacctcaaggcattgcccctgttcctccacctagggacctaagctgggaagcaggacatggaggaaatgtggctttgccacttaaggagacatccgtggaaaactttctgggagctaccacacctgccctggctaagctgcaggtgccgcgggagcaactctctgaagttctggagcagatttgcagcggtagctgtgggcccatctttcgagccaatatgaacactggggacccttctaagcccaagagtgttattctcaaggctttaaaagaaccagctgggctccatgaggtacaagatttcttagggcgaatccaattccatcaatacctggggaaacacaaaaacctggtgcagctggaaggctgctgcactgaaaagctgccactctatgtggtgttggaggatgtggcccagggggacctgcttagctttctctggacctgtcggcgggatgtgatgactatggatggtcttctctatgatctcacagaaaaacaagtatatcacatcggaaagcaggtccttttggcgctggaattcctgcaggagaagcatttgttccatggggatgtggcagccaggaatattctgatgcaaagtgatctcactgctaagctctgtggattaggcctggcttatgaagtttacacccgaggggccatctcctctactcaaaccatacctctcaagtggcttgccccagaacggcttctcctgagacctgctcgcatcagagcagatgtctggtcttttgggatcctgctctatgagatggtgactctaggagcaccaccgtatcctgaagtccctcctaccagcatcctagagcatctccaaagaaggaaaatcatgaagagacccagtagctgcacacataccatgtacagtatcatgaagtcctgctggcgctggcgtgaggctgaccgcccctcacctagagagctgcgcttgcgcctagaagctgccattaaaactgcagatgacgaggctgtgttacaagtaccagagttggtggtacctgaactgtatgcagctgtggccggcatcagagtggagagcctcttctacaactatagcatgctttacccctacgacgtgcccgactacgcctga

SEQ ID NO: 4 - protein, 431 aa, Homo sapiens
MGMTRMLLECSLSDKLCVIQEKQYEVIIVPTLLVTIFLILLGVILWLFIREQRTQQQRSGPQGIAPVPPPRDLSWEAGHGGNVALPLKETSVENFLGATTPALAKLQVPREQLSEVLEQICSGSCGPIFRANMNTGDPSKPKSVILKALKEPAGLHEVQDFLGRIQFHQYLGKHKNLVQLEGCCTEKLPLYVVLEDVAQGDLLSFLWTCRRDVMTMDGLLYDLTEKQVYHIGKQVLLALEFLQEKHLFHGDVAARNILMQSDLTAKLCGLGLAYEVYTRGAISSTQTIPLKWLAPERLLLRPARIRADVWSFGILLYEMVTLGAPPYPEVPPTSILEHLQRRKIMKRPSSCTHTMYSIMKSCWRWREADRPSPRELRLRLEAAIKTADDEAVLQVPELVVPELYAAVAGIRVESLFYNYSMLYPYDVPDYA

SEQ ID NO: 5 - DNA, 1953 bp, Homo sapiens
  CDS (1)..(1953)
atggacaaactcagggtgcccctctggcctcgggtaggccccctctgtctcctacttgctggggcagcctgggcaccttcacccagcctcccggaccccaagtttgagagcaaagcggccctgctggcatcccggggctccgaagaacttctgtgcttcacccaacgcttggaagacttggtgtgtttctgggaggaagcggcgagctccgggatggacttcaactacagcttctcataccagctcgagggtgagtcacgaaagtcatgtagcctgcaccaggctcccaccgtccgcggctccgtgcgtttctggtgttcactgccaacagcggacacatcgagttttgtgccgctggagctgcaggtgacggaggcgtccggttctcctcgctatcaccgcatcatccatatcaatgaagtagtgctcctggacgcccccgcggggctgctggcgcgccgggcagaagagggcagccacgtggtgctgcgctggctgccacctcctggagcacctatgaccacccacatccgatatgaagtggacgtgtcggcaggcaaccgggcaggagggacacaaagggtggaggtcctggaaggccgcactgagtgtgttctgagcaacctgcggggcgggacgcgctacaccttcgctgttcgagcgcgcatggccgagccgagcttcagcggattctggagtgcctggtctgagcccgcgtcactactgaccgctagcgacctggaccctctcgcggccgcagtgattatcgtcccaactttgttggttactatcttcctcatccttcttggggtcatcctgtggctttttatcagagaacaaagaactcaacagcagcgttctggacctcaaggcattgcccctgttcctccacctagggacctaagctgggaagcaggacatggaggaaatgtggctttgccacttaaggagacatccgtggaaaactttctgggagctaccacacctgccctggctaagctgcaggtgccgcgggagcaactctctgaagttctggagcagatttgcagcggtagctgtgggcccatctttcgagccaatatgaacactggggacccttctaagcccaagagtgttattctcaaggctttaaaagaaccagctgggctccatgaggtacaagatttcttagggcgaatccaattccatcaatacctggggaaacacaaaaacctggtgcagctggaaggctgctgcactgaaaagctgccactctatgtggtgttggaggatgtggcccagggggacctgcttagctttctctggacctgtcggcgggatgtgatgactatggatggtcttctctatgatctcacagaaaaacaagtatatcacatcggaaagcaggtccttttggcgctggaattcctgcaggagaagcatttgttccatggggatgtggcagccaggaatattctgatgcaaagtgatctcactgctaagctctgtggattaggcctggcttatgaagtttacacccgaggggccatctcctctactcaaaccatacctctcaagtggcttgccccagaacggcttctcctgagacctgctcgcatcagagcagatgtctggtcttttgggatcctgctctatgagatggtgactctaggagcaccaccgtatcctgaagtccctcctaccagcatcctagagcatctccaaagaaggaaaatcatgaagagacccagtagctgcacacataccatgtacagtatcatgaagtcctgctggcgctggcgtgaggctgaccgcccctcacctagagagctgcgcttgcgcctagaagctgccattaaaactgcagatgacgaggctgtgttacaagtaccagagttggtggtacctgaactgtatgcagctgtggccggcatcagagtggagagcctcttctacaactatagcatgctttga

SEQ ID NO: 6 - protein, 650 aa, Homo sapiens
MDKLRVPLWPRVGPLCLLLAGAAWAPSPSLPDPKFESKAALLASRGSEELLCFTQRLEDLVCFWEEAASSGMDFNYSFSYQLEGESRKSCSLHQAPTVRGSVRFWCSLPTADTSSFVPLELQVTEASGSPRYHRIIHINEVVLLDAPAGLLARRAEEGSHVVLRWLPPPGAPMTTHIRYEVDVSAGNRAGGTQRVEVLEGRTECVLSNLRGGTRYTFAVRARMAEPSFSGFWSAWSEPASLLTASDLDPLAAAVIIVPTLLVTIFLILLGVILWLFIREQRTQQQRSGPQGIAPVPPPRDLSWEAGHGGNVALPLKETSVENFLGATTPALAKLQVPREQLSEVLEQICSGSCGPIFRANMNTGDPSKPKSVILKALKEPAGLHEVQDFLGRIQFHQYLGKHKNLVQLEGCCTEKLPLYVVLEDVAQGDLLSFLWTCRRDVMTMDGLLYDLTEKQVYHIGKQVLLALEFLQEKHLFHGDVAARNILMQSDLTAKLCGLGLAYEVYTRGAISSTQTIPLKWLAPERLLLRPARIRADVWSFGILLYEMVTLGAPPYPEVPPTSILEHLQRRKIMKRPSSCTHTMYSIMKSCWRWREADRPSPRELRLRLEAAIKTADDEAVLQVPELVVPELYAAVAGIRVESLFYNYSML

SEQ ID NO: 7 - DNA, 1977 bp, Homo sapiens
  CDS (1)..(1977)
atggacaaactcagggtgcccctctggcctcgggtaggccccctctgtctcctacttgctggggcagcctgggcaccttcacccagcctcccggaccccaagtttgagagcaaagcggccctgctggcatcccggggctccgaagaacttctgtgcttcacccaacgcttggaagacttggtgtgtttctgggaggaagcggcgagctccgggatggacttcaactacagcttctcataccagctcgagggtgagtcacgaaagtcatgtagcctgcaccaggctcccaccgtccgcggctccgtgcgtttctggtgttcactgccaacagcggacacatcgagttttgtgccgctggagctgcaggtgacggaggcgtccggttctcctcgctatcaccgcatcatccatatcaatgaagtagtgctcctggacgcccccgcggggctgctggcgcgccgggcagaagagggcagccacgtggtgctgcgctggctgccacctcctggagcacctatgaccacccacatccgatatgaagtggacgtgtcggcaggcaaccgggcaggagggacacaaagggtggaggtcctggaaggccgcactgagtgtgttctgagcaacctgcggggcgggacgcgctacaccttcgctgttcgagcgcgcatggccgagccgagcttcagcggattctggagtgcctggtctgagcccgcgtcactactgaccgctagcgacctggaccctctcgcggccgcagtgattatcgtcccaactttgttggttactatcttcctcatccttcttggggtcatcctgtggctttttatcagagaacaaagaactcaacagcagcgttctggacctcaaggcattgcccctgttcctccacctagggacctaagctgggaagcaggacatggaggaaatgtggctttgccacttaaggagacatccgtggaaaactttctgggagctaccacacctgccctggctaagctgcaggtgccgcgggagcaactctctgaagttctggagcagatttgcagcggtagctgtgggcccatctttcgagccaatatgaacactggggacccttctaagcccaagagtgttattctcaaggctttaaaagaaccagctgggctccatgaggtacaagatttcttagggcgaatccaattccatcaatacctggggaaacacaaaaacctggtgcagctggaaggctgctgcactgaaaagctgccactctatgtggtgttggaggatgtggcccagggggacctgcttagctttctctggacctgtcggcgggatgtgatgactatggatggtcttctctatgatctcacagaaaaacaagtatatcacatcggaaagcaggtccttttggcgctggaattcctgcaggagaagcatttgttccatggggatgtggcagccaggaatattctgatgcaaagtgatctcactgctaagctctgtggattaggcctggcttatgaagtttacacccgaggggccatctcctctactcaaaccatacctctcaagtggcttgccccagaacggcttctcctgagacctgctcgcatcagagcagatgtctggtcttttgggatcctgctctatgagatggtgactctaggagcaccaccgtatcctgaagtccctcctaccagcatcctagagcatctccaaagaaggaaaatcatgaagagacccagtagctgcacacataccatgtacagtatcatgaagtcctgctggcgctggcgtgaggctgaccgcccctcacctagagagctgcgcttgcgcctagaagctgccattaaaactgcagatgacgaggctgtgttacaagtaccagagttggtggtacctgaactgtatgcagctgtggccggcatcagagtggagagcctcttctacaactatagcatgcttgactacaaggacgacgatgacaagtga

SEQ ID NO: 8 - protein, 658 aa, Homo sapiens
MDKLRVPLWPRVGPLCLLLAGAAWAPSPSLPDPKFESKAALLASRGSEELLCFTQRLEDLVCFWEEAASSGMDFNYSFSYQLEGESRKSCSLHQAPTVRGSVRFWCSLPTADTSSFVPLELQVTEASGSPRYHRIIHINEVVLLDAPAGLLARRAEEGSHVVLRWLPPPGAPMTTHIRYEVDVSAGNRAGGTQRVEVLEGRTECVLSNLRGGTRYTFAVRARMAEPSFSGFWSAWSEPASLLTASDLDPLAAAVIIVPTLLVTIFLILLGVILWLFIREQRTQQQRSGPQGIAPVPPPRDLSWEAGHGGNVALPLKETSVENFLGATTPALAKLQVPREQLSEVLEQICSGSCGPIFRANMNTGDPSKPKSVILKALKEPAGLHEVQDFLGRIQFHQYLGKHKNLVQLEGCCTEKLPLYVVLEDVAQGDLLSFLWTCRRDVMTMDGLLYDLTEKQVYHIGKQVLLALEFLQEKHLFHGDVAARNILMQSDLTAKLCGLGLAYEVYTRGAISSTQTIPLKWLAPERLLLRPARIRADVWSFGILLYEMVTLGAPPYPEVPPTSILEHLQRRKIMKRPSSCTHTMYSIMKSCWRWREADRPSPRELRLRLEAAIKTADDEAVLQVPELVVPELYAAVAGIRVESLFYNYSMLDYKDDDDK

SEQ ID NO: 9 - DNA, 63 bp, Artificial
  epitope
aagtcctgctggcgctggcgtgaggctgaccgcccctcacctagagagctgcgcttgcgccta

SEQ ID NO: 10 - protein, 21 aa, Artificial
  Synthetic Construct
KSCWRWREADRPSPRELRLRL

SEQ ID NO: 11 - DNA, 33 bp, Artificial
  primer
tataaagcttatgggcatgatgacacggatgct

SEQ ID NO: 12 - DNA, 58 bp, Artificial
  primer
tatactcgagtcaggcgtagtcgggcacgtcgtaggggtaaagcatgctatagttgta

SEQ ID NO: 13 - DNA, 32 bp, Artificial
  primer
tatagcgatatcatggacaaactcagggtgcc

SEQ ID NO: 14 - DNA, 33 bp, Artificial
  primer
tatagcgcggccgcgagagggtccaggtcgcta

SEQ ID NO: 15 - DNA, 33 bp, Artificial
  primer
tatagcggccgcagtgattatcgtcccaacttt

SEQ ID NO: 16 - DNA, 63 bp, Artificial
  primer
tataccagtgtgctggtcacttgtcatcgtcgtccttgtagtcaagcatgctatagttgtaga

SEQ ID NO: 17 - DNA, 22 bp, Artificial
  primer
cctcctaccagcatcctagagc

SEQ ID NO: 18 - DNA, 25 bp, Artificial
  primer
gcacacataccatgttcagtatcat

SEQ ID NO: 19 - DNA, 22 bp, Artificial
  primer
gacttcaggaaacggtggtgct

SEQ ID NO: 20 - DNA, 25 bp, Artificial
  primer
agctactgggtctcttcatgatttt

SEQ ID NO: 21 - DNA, 30 bp, Artificial
  primer
tggcccgcctggcattatgcccagtacatg

SEQ ID NO: 22 - DNA, 30 bp, Artificial
  primer
agccacaggatgaccccaagaaggatgagg

SEQ ID NO: 23 - DNA, 30 bp, Artificial
  primer
tcctgaagtccctcctaccagcatcctaga

SEQ ID NO: 24 - DNA, 30 bp, Artificial
  primer
tcttcccaatcctcccccttgctgtcctgc

SEQ ID NO: 25 - DNA, 20 bp, Artificial
  primer
atgggcatgacacggatgct

SEQ ID NO: 26 - DNA, 19 bp, Artificial
  primer
tcaaagcatgctatagttg

(19) United States
(12) Patent Application Publication
(10) Pub. No.: US 2009/0222931 A1
(43) Pub. Date: Sep. 3, 2009

(54) NOVEL IDENTIFIED ONCOGENE WITH KINASE-DOMAIN (NOK)

(75) Inventors: Li Liu, Beijing (CN); Xinyuan Fu, Indianapolis, IN (US); Zhijie Chang, Beijing (CN); Shuping Zhang, Beijing (CN)

Correspondence Address: HOWREY LLP - East, C/O IP DOCKETING DEPARTMENT, 2941 FAIRVIEW PARK DR, SUITE 200, FALLS CHURCH, VA, 22042-2924, US

(73) Assignee: TSINGHUA UNIVERSITY, Beijing (CN)

(21) Appl. No.: 11/568,972

(22) PCT Filed: May 13, 2004
(86) PCT No.: PCT/CN05/00669

§ 371 Date: May 14, 2009

(30) Foreign Application Priority Data
May 13, 2004 (CN) 200410037888.2
May 13, 2004 (CN) 200410037889.7

(51) Int. Cl. C12N 9/12; C12N 15/11; C12N 15/85; C12N 5/10; C12N 9/96; C12P 21/02; C12P 21/04; C07K 16/18; C07H 21/04; A01K 67/027; C12Q 1/02; G01N 33/00; C12Q 1/68

(52) U.S. Cl. 800/3; 536/23.2; 435/320.1; 435/325; 435/194; 435/188; 435/69.1; 435/69.7; 530/387.7; 536/24.31; 536/24.33; 800/13; 435/29; 435/6

(57) ABSTRACT

A newly identified oncogene with kinase-domain (NOK) and its encoded polypeptide, and vectors, fusions, host cells and transgenic animals comprising the said nucleotide sequence. Furthermore, the present invention also describes the methods for diagnosing diseases including tumor and the methods for screening agents capable of inhibiting the occurrence and/or metastasis of tumor.

### FIELD OF INVENTION

The present invention relates generally to the field of tumor biology, and more specifically to an oncogene and the protein encoded by the same. The inventors named this oncogene as novel oncogene with kinase-domain, and used the abbreviated name “NOK” in the following text.

### BACKGROUND OF INVENTION

Receptor protein tyrosine kinases (RPTKs) play important roles in diverse cellular regulations and developmental processes, such as cell proliferation, differentiation and survival (Hunter T. Signaling: 2000 and beyond. Cell 2000; 100: 113-27; Schlessinger J. Cell signaling by receptor tyrosine kinases. Cell 2000; 103: 211-25.). The typical structure of RPTK is a single transmembrane protein consisting of an extracellular domain, a transmembrane domain and an intracellular domain. The extracellular domain contains a specific ligand binding site and the intracellular domain contains a tyrosine kinase domain which is involved in activating downstream signaling cascades (Blume-Jensen P, Hunter T. Oncogenic kinase signalling. Nature 2001; 411:355-65). RPTKs are often involved in mitogenic signaling, therefore, stringent regulation of RPTK expression is required for maintaining the normal cellular functions (Hubbard S R, et al. Autoregulatory mechanisms in protein tyrosine kinases. J Biol Chem 1998; 273: 11987-90.). In contrast, aberrant expressions and activation of RPTK can cause numerous genetic disorders including tumor formation (Powers C J, et al. Fibroblast growth factors, their receptors and signaling. Endocr Relat Cancer 2000; 7:165-97). At present, at least 18 RPTKs have been demonstrated to function as oncogenes (Blume-Jensen P, Hunter T. Oncogenic kinase signalling. Nature 2001; 411:355-65). The well-known examples include fibroblast growth factor receptor (FGFR), epidermal growth factor receptor (EGFR), platelet-derived growth factor receptor (PDGFR) and MET/Ron tyrosine kinase receptor etc.

Aberrant expressions of certain spliced variants of RPTK have frequently been documented and implicated in numerous human cancers. Soluble FGFR3 lacking the entire transmembrane domain has been isolated from human osteosarcoma and breast cancer cells (Johnston C L, et al. Fibroblast growth factor receptors (FGFRs) localize in different cellular compartments: a spliced variant of FGFR-3 localizes to the nucleus. J Biol Chem 1995; 270: 30643-50; Jang J H. Identification and characterization of soluble isoform of fibroblast growth factor receptor 3 in human SaOS-2 osteosarcoma cells. Biochem Biophys Res Commun 2002; 292:378-82). An in-frame deletion of 49 amino acids in the extracellular domain of the Ron tyrosine kinase receptor leads to constitutive receptor activation in human gastric cancer cell line (KATO-III) (Collesi C, et al. A spliced variant of the RON transcript induces constitutive tyrosine kinase activity and an invasive phenotype. Mol Cell Biol 1996; 16:5518-26.). Most recently, a novel splice variant of FGFR4 (ptd-FGFR4) was isolated from human pituitary tumor (Ezzat S, et al. Targeted expression of a human pituitary tumor-derived isoform of FGF receptor-4 recapitulates pituitary tumorigenesis. J Clin Investig 2002; 109:69-78). Intriguingly, this ptd-FGFR4 is N-terminally truncated at the upstream of IgIIIc domain resulting in an intracellular FGFR4 variant without 5′ signal peptide, IgI and IgII. As a result of the truncation, this protein was exclusively retained in the cytoplasm compartment. ptd-FGFR4 was constitutively active when it was stably expressed in NIH3T3 cells and caused cellular transformation and tumor formation in nude mice. Moreover, selective expression of ptd-FGFR4 in transgenic mice recapitulated pituitary tumor progression in human. Another example of receptors with N-terminal truncation is the hepatocyte growth factor receptor (Met). N-terminal truncated Met has been implicated in the human malignant musculoskeletal tumors (Wallenius V, et al. Overexpression of the hepatocyte growth factor (HGF) receptor (Met) and presence of a truncated and activated intracellular HGF receptor fragment in locally aggressive/malignant human musculoskeletal tumors. Am J Pathol 2000; 156: 821-9).

### SUMMARY OF INVENTION

The inventors identified and cloned an oncogene with a typical kinase domain. This gene has significant homology with the members of FGFR/PDGFR superfamily at both nucleotide and amino acid levels. The inventors named this oncogene as novel oncogene with kinase-domain (NOK).

The present invention provides an isolated polynucleotide comprising a nucleotide sequence selected from:

1) the nucleotide sequence of SEQ ID NO: 1;
2) the nucleotide sequence encoding the amino acid sequence of SEQ ID NO: 2; and
3) a nucleotide sequence with at least 90% sequence identity with that of 1) and 2), and the isolated polynucleotide encodes a mammal NOK gene product.

The present invention also provides an isolated polynucleotide encoding a chimeric polypeptide that is fused between NOK and at least one heterogenous polypeptide.

Preferably, the isolated polynucleotide of the invention encoding the chimeric polypeptide comprises a nucleotide sequence selected from:

1) the nucleotide sequence of SEQ ID NO:5
2) the nucleotide sequence encoding the amino acid sequence of SEQ ID NO:6, and
3) a nucleotide sequence that has at least 90% identity with 1) or 2).

The present invention provides an expression vector that contains the polynucleotide of the invention.

The present invention also provides host cells transformed with an expression vector that contains the polynucleotide of the invention.

The present invention further provides an isolated polypeptide comprising the amino acid sequence of SEQ ID NO:2 or a biologically active fragment or derivative thereof. The derivative of polypeptide of the invention has an amino acid sequence of SEQ ID NO:2 with substitution, deletion or insertion of one or several amino acids and has the same biological function(s) of SEQ ID NO:2.

The present invention further provides a fusion polypeptide that is a chimeric molecule formed between NOK and at least one heterogenous polypeptide.

Preferably, the fusion polypeptide of present invention is a chimeric receptor (EPOR/NOK) formed between the extracellular domain of mouse erythropoietin receptor (EPOR) and the transmembrane and intracellular domain of human NOK gene.

In another aspect, the present invention provides a method of producing the polypeptide or the fusion polypeptide of the invention. The method includes the steps of culturing the host cells of the invention under conditions suitable for the expression and purification of said polypeptide, and collecting said polypeptide.

In a further aspect, the present invention provides a polypeptide epitope corresponding to the 360^{th} to 380^{th}, amino acid residues of the amino acid sequence of NOK. The present invention also provides a nucleotide sequence that encodes the said polypeptide epitope.

The present invention also provides an antibody that can specifically bind to the polypeptide of the invention. Preferably, the antibody can specifically bind to the epitope of the above polypeptide of the invention.

The present invention further provides a mutant of EPOR/NOK comprising a single point mutation at either tyrosine 327 or 356 in the NOK moiety of the EPOR/NOK fusion protein.

The present invention also provides an oligonucleotide or primer that can hybridize with the polynucleotide of the invention.

In yet another aspect, the present invention provides a transgenic animal harboring the polynucleotide of the invention that encodes the protein product of the novel oncogene with kinase domain. Preferably, the transgenic animal of the present invention is mouse. The transgenic animal of the invention provides a useful model system to study the mechanisms of tumorigenesis or a useful tool for the development of anti-tumor therapy. The present invention thus also relates to a method for screening an agent with anti-tumor growth and/or anti-metastasis activities, which method comprises the step of determining the inhibitory effects of a candidate agent on the tumor growth and/or matastasis in the transgenic mice of the invention. According to the present invention, another method for screening an agent with anti-tumor growth and/or anti-metastasis activities includes the step of determining the inhibitory effects of a candidate agent on the proliferation of the host cells of the invention that have been transformed with NOK, or on the tumor growth and/or metastasis in nude mice that have been inoculated with the cell line of the invention.

The present invention also relates to a method for detecting the presence of a disease or the susceptibility to a disease in a subject, comprising the step of detecting the presence of the polynucleotide or polypeptide of the invention or a mutant thereof in a biological sample.

The present invention also relates to a clinical diagnostic kit that contains an antibody or an oligonucleotide probe or primer of the invention.

The present invention will be illustrated in more detail with reference to the following drawings and non-limiting examples.

### BRIEF DESCRIPTION OF THE DRAWINGS

FIG. 1. The NOK gene products obtained by RT-PCR amplification using total RNA prepared from human amygdala tissue.

FIG. 2. Western blot analysis of NOK protein expression in BaF3-p3 and BaF3-NOK cells by using anti-HA as a primary antibody.

FIG. 3. The proliferation curve of BaF3-NOK cells at starvation condition (without WEHI-3B conditioned medium and serum).

FIG. 4. Colony formation of BaF3-NOK cells in soft agar at starvation condition (without WEHI-3B conditioned medium and serum).

FIG. 5. Tumor formation in nude mice after s.c. injections of BaF3-NOK cells.

FIG. 6. The metastasis of BaF3-NOK cells into distant organs in nude mice such as liver, spleen and kidney, and the penetration of tumor cells in the skeletal muscle at the injection site.

FIG. 7. Transmembrane analysis of EPOR/NOK chimeric receptor by Dense Alignment Surface (DAS) program.

FIG. 8. Structural analysis of the protein tyrosine kinase domain of EPOR/NOK.

FIG. 9. Western blot analysis of EPOR/NOK protein expression in BaF3-p3 and BaF3-EPOR/NOK cells by using mouse anti-FLAG antibody.

FIG. 10. The proliferation curve of BaF3-EPOR/NOK cells at starvation condition (without WEHI-3B conditioned medium and serum).

FIG. 11. Colony formation of BaF3-EPOR/NOK cells in soft agar at starvation condition (without WEHI-3B conditioned medium and serum).

FIG. 12. Tumor formation in nude mice after s.c. injections of BAF3-EPOR/NOK cells.

FIG. 13. Haematoxylin & Eosin (HE) Staining shows the metastasis of BaF3-NOK tumor cells in distant organs.

FIG. 14. The hydrophobic analysis of NOK protein by Kyte-Doolittle.

FIG. 15. The secondary structure of NOK protein predicted with nnPredict method.

FIG. 16. Western blot analysis of NOK protein expression by using the antibody generated by using the predicted NOK epitope.

FIG. 17. Immunohistochemistical analysis on the liver section of BaF3-EPOR NOK injected nude mouse by using polyclonal rabbit anti-NOK antibody.

FIG. 18. Comparison of sequence homology between the intracellular domains of NOK and other 9 protein receptor tyrosine kinases by Sequence Alignment (ClustalW Software).

FIG. 19. [3H] thymidine incorporation assay on BaF3-EPOR/NOK and its mutant derivatives.

FIG. 20. Colony formation assay on BaF3-EPOR/NOK and its mutant derivatives.

FIG. 21. Tumor formation assay by inoculating BaF3-EPOR/NOK and its mutant derivatives into nude mice.

FIG. 22. The survival curve of nude mice that has been subcutaneously injected with BaF3-EPOR/NOK and its mutant derivatives.

FIG. 23. Haematoxylin & Eosin (HE) staining of different organs of nude mice that have been subcutaneously injected with BaF3-EPOR/NOK and its mutant derivatives.

FIG. 24. In vitro kinase assay of the chimeric receptor EPOR/NOK and its mutant derivatives.

FIG. 25. The ERK activities of EPOR/NOK and its mutant derivatives in BaF3 stable cells.

FIG. 26. The AKT activities of EPOR/NOK and its mutant derivatives in BaF3 stable cells.

FIG. 27. The STAT5 activities of EPOR/NOK and its mutant derivatives in BaF3 stable cells.

FIG. 28. NOK represses the expression of E-cadherin.

FIG. 29. Genomic PCR of the NOK transgenic mice.

FIG. 30. Western blot analysis of the tissue distribution of NOK gene expression in NOK transgenic mice.

FIG. 31. The expression profile of NOK mRNAs in different lymphoid organs in wild type and NOK transgenic mice.

FIG. 32. Enlargement of peripheral lymph nodes in NOK transgenic mice.

FIG. 33. The metastatic foci formed by lymphoid cells in different organs of NOK transgenic mice.

FIG. 34. Immunohistochemistical analysis of major organs of NOK transgenic mice using the NOK specific antibody.

FIG. 35. Detection of NOK gene expression in liver sections of nude mouse that was injected with the tumor cell prepared from lymph node of NOK transgenic mouse.

FIG. 36. POX staining of the peripheral blood smear prepared from a typical transgenic mouse.

FIG. 37. Flow cytometry analysis of the IgM+ B lymphocytes in the lymph node of the wild type and NOK transgenic mice.

FIG. 38. Flow cytometry analysis of the CD19+/CD22+ B lymphocytes prepared from the peripheral lymphoid organs of NOK transgenic mice.

FIG. 39. Flow cytometry analysis of IgM+ B lymphocytes from the peripheral blood and of CD19+/CD22+ or CD79α+ B lymphocytes from the lymph node of nude mouse that was inoculated with cell suspension prepared from the lymph nodes of NOK transgenic mice.

FIG. 40. The results of the tumor microarray analysis on head and neck cancers by using rabbit polyclonal anti-NOK antibody.

FIG. 41. High levels of NOK protein expressions detected in various types of tumor tissues, including thyroid carcinoma, skin cancer, colon cancer, rectum cancer et al.

### DEPOSIT

The BaF3-NOK stable cell used in the present invention was deposited in China General Microbiological Culture Collection Center (CGMCC) on May 9, 2004 with the deposit number of CGMCC No. 1145.

The BaF3-EPOR/NOK stable cell used in the present invention was deposited in China General Microbiological Culture Collection Center (CGMCC) on May 9, 2004 with the deposit number of CGMCC No. 1144.

### DETAILED DESCRIPTION OF THE INVENTION

The inventors have obtained and functionally characterized a novel gene encoding a receptor protein tyrosine kinase-like (RPTK-like) molecule that has a typical kinase domain. This RPTK-like molecule has significant homology with the members of FGFR/PDGFR superfamily at both nucleotide and amino acid levels (with 22-23% amino acid identity). The gene of the invention encodes a transmembrane protein with 422 amino acids in length. This molecule has a typical tyrosine kinase domain but does not have a signal peptide and an extracellular domain. The results of functional characterization presented in the experimental section of the invention demonstrate that the gene of the invention functions as an oncogene that can stimulate multiple mitogenic signaling pathways, transform both murine pro-B cell line (BaF3) and murine fibroblast cell line (NIH3T3) cells, and induce tumorigenesis and metastasis in animal model. Based on these experimental results, the gene encoding the novel RPTK molecule of the present invention was designated as a novel oncogene with kinase-domain (NOK).

The present invention thus provides an isolated polynucleotide that encodes a novel oncogene with kinase domain, NOK. The isolated polynuceotide that encodes NOK protein comprises a nucleotide sequence selected from:
1) the nucleotide sequence of SEQ ID NO: 1□
2) a nucleotide sequence encoding the amino acid sequence of SEQ ID NO:2; and
3) a nucleotide sequence that has at least 90% sequence identity with that of 1) or 2), and encodes a protein with the same function as NOK protein.

SEQ ID NO:1 is consisted of 1269 bases. The open reading frame of this gene starts from its first nucleotide at the 5′ end and terminate at the 1269th nucleotide. In an embodiment of the invention, a nucleotide sequence encoding an HA tag is added to the 3′ end of the gene to facilitate the detection of the gene expression. The complete nucleotide sequence of the HA-tagged coding sequence is shown in SEQ ID NO:3. SEQ ID NO:3 is consisted of 1296 bases, and the coding sequence of HA tag is localized between 1267th and 1296th nucleotides in 5′ to 3′ direction.

In this application, the term “isolated polynucleotide” means a polynucleotide that has been purified or separated from polynucleotides to which it is associated or linked in its natural state. Preferably, the isolated polynucleotide has been purified or separated to an extent of at least 70%, more preferably to an extent of at least 80%, and most preferably to an extent of at least 90%, from polynucleotides to which it is associated or linked in its natural state. In this text, the terms “polynucleotide”, “nucleic acid molecule”, and “gene” can be used interchangeably.

A polynucleotide of the invention may have one or several mutations, as compared with the nucleotide sequence specifically provided in the sequence listing for the polynucleotide of the invention. Such mutations can be deletion, insertion or substitution of one or several nucleotides. The mutant may be either a naturally occurring one (e.g., isolated from a natural source) or a synthesized one (e.g., generated through site directed mutagenesis). Therefore, the polynucleotide of the present invention can be either a naturally occurring molecule or a recombinant molecule.

The present invention further provides an isolated polypeptide comprising the amino acid sequence of SEQ ID NO:2 or a biologically active fragment or derivative thereof. The polypeptide of SEQ ID NO:2 is consisted of 422 amino acid residues. The derivative of the polypeptide of present invention comprises an amino acid sequence of SEQ ID NO:2 with substitution, deletion, or insertion of one or several amino acids, and has the same biological activity as SEQ ID NO:2.

In this context, the terms “isolated polypeptide” means a polypeptide that has been extracted from lipids, nucleic acids, other polypeptides and other molecules to which it is associated in its natural state. Preferably, the isolated polypeptide has been purified or separated to an extent of at least 70%, more preferably to an extent of at least 80%, and most preferably to an extent of at least 90%, from the components to which it is associated in its natural state. In this text, the terms “peptide”, “polypeptide”, and “protein” can be used interchangeably.

It can be understood that the mutants of the polypeptide of the invention can be obtained by appropriate nucleotide changes in the coding sequence or by in vitro synthesis. The mutation includes, for example, deletion, insertion or substitution in the amino acid sequence. The final construct can be obtained by the combination of these two or three approaches, provided the protein product still has the desired properties. The present invention also encompasses derivatives of the polypeptide of the invention that can be obtained by modifications such as (but not limited to) biotinylation, benzylation, glycosylation, acetylation, phosphorylation, aminoacylation, derivatization with known protective/inhibitory group, protein hydrolysis, and ligation with antibody or cellular ligand and the like. These modifications can either enhance or decrease the stability and/or biological activity of the polypeptide of the invention.

In a particular embodiment, in order to facilitate the detection, the carboxyl terminus of the polypeptide of the invention carries a HA tag. This HA-tagged NOK amino acid sequence is shown in SEQ ID NO:4. SEQ ID NO:4 is consisted of 431 amino acids in which the HA tag is located from the amino acid 423rd residue to 431 st residue.

In another aspect, the present invention provides an expression vector comprising the polynucleotide encoding NOK.

The expression vector of the invention comprises an isolated polynucleotide of the invention. The vector can be any proper vector that is able to carry and deliver the inserted polynucleotide into a host cell. The vector may comprise heterogeneous nucleic acid sequences. “Heterogeneous nucleic acid” means a polynucleotide sequence that is not adjacent to the polynucleotide of the invention in natural state. The vector may be either RNA or DNA vector, a prokaryotic or eukaryotic vector, and typically, a DNA plasmid.

The type of the expression vector comprises the polynucleotide of the invention operably linked within the expression vector. By “operably linked”, it means that a polynucleotide is inserted and linked in the expression vector in such a way that it can be expressed upon the vector being transformed/transfected into the host cells. The expression vector is a DNA or RNA vector that, upon transformed/transfected into the host cell, allows the expression of a particular polynucleotide in the host cell. Preferably, the expression vector is able to replicate in the host cell. The expression vector can be either a prokaryotic or eukaryotic vector, and is typically a plasmid or virus. The expression vector of the invention includes any vector that functions (capable of directing gene expression) in the recombinant host cells of the invention. The recombinant host cells include bacterial, yeast, and mammalian cells. Preferably, the expression vector of the invention is able to direct gene expression in bacterial, yeast, and mammalian cells.

The expression vector of the invention contains regulatory sequences such as transcriptional regulatory sequence, translational regulatory sequence, replication initiation site, and other regulatory sequences that are compatible with the recombinant cell lines and can direct the gene expression of the polynucleotide of the invention. Particularly, the recombinant molecule comprises transcriptional regulatory sequences that control the initiation, enlongation, and termination of transcription. Important transcriptional regulatory sequences include those sequences that regulate the transcriptional initiation such as promoter, enhancer, operon and repressor sequences. The transcriptional regulatory sequences suitable for the present invention include any regulatory sequence that can function in at least one of the host cell of the invention. Such regulatory sequences are well known for those skilled in the art.

In a particular embodiment, the vector of the invention comprising a polynucleotide encoding NOK is the expression vector pcDNA3.0(NOK) as shown in Example 1.

The recombinant molecule of the invention (a) may comprise a secretory signal (i.e., the nucleotide sequence encoding a signal peptide) which allows the secretion of the polypeptide of the invention produced in the host cells from the host cells; and/or (b) may be a fusion sequence that allows the polypeptide of the invention to be expressed as a chimeric protein. Appropriate signal peptides include any signal fragment that directs the secretion of the protein of the invention.

In another aspect, the present invention provides a host cell that has been transformed with a vector comprising the polynucleotide encoding NOK.

The polynucleotide of the invention can be transformed into the host cells by any technique that can effectively deliver a polynucleotide into the host cells. The technique includes (but not limited to) transfection, electroporation, microinjection, lipofectin, and viral infection. The transformed host-cells not only can be maintained in a single cell state, but also can grow in animal tissues or organs as well as multi cell organisms. The polynucleotide of the invention that has been delivered into host cells can be either maintained extra-chromosomally or integrated into host genome at one or more locations to ensure its expression capacity.

The host cells that are suitable for the present invention include any cells that can be transformed by the polynucleotide of the invention. The host cell can be either an un-transformed cell or a cell that has been already transformed by at least one polynucleotide (for example, polynucleotide(s) encoding one or more than one proteins of the invention). The host cell of the invention may produce the desired protein endogenously (naturally), or may generate the desired protein after transformed with at least one of the polynucleotide of the invention.

In a particular embodiment, the host cell of the present invention that has been transformed with the polynucleotide encoding NOK is the BaF3-NOK cell line. This cell line was deposited at China General Microbiological Culture Collection Center (CGMCC) on May 9, 2004 with the deposit number of CGMCC No. 1145.

The inventors found that stable expression of NOK gene in BaF3 cells resulted in transformation of the BaF3 cells, and the growth of BaF3 cells changed from an IL-3 dependent pattern to an IL-3 independent pattern. Injection (s.c.) of BaF3-NOK cells stably expressing NOK into nude mice resulted in not only tumor formation at the injection site but also metastasis at multiple distant organs, which is the typical manifestations of malignant tumor. Thus, this supports NOK can be defined as a novel oncogene. BaF3-NOK injected nude mice can serve as a model system not only for the study of the mechanisms of tumorigenesis and metastasis, but also for screening an agent with anti-tumorigenesis and anti-metastasis activities. In addition, BaF3-NOK cell also provides a good cellular model system for screening and evaluating an anti-tumor agent against NOK-induced tumorigenesis and metastasis.

The present invention provides a fusion polypeptide which is a chimeric molecule formed between NOK and at least one heterogeneous polypeptide. In a particular embodiment, the fusion polypeptide is a chimeric receptor EPOR/NOK that is formed by fusing the extracellular domain of mouse erythropoietin receptor (EPOR) with the transmembrane and intracellular domains of human NOK.

The EPOR/NOK chimeric receptor is a protein comprising the amino acid sequence of SEQ ID NO:6, or a protein comprising an amino acid sequence derived from SEQ ID NO:6 by one or several substitution, deletion, or insertion in the amino acid sequences of SEQ ID NO:6 and having the same activities as SEQ ID NO:6.

SEQ ID NO:6 is consisted of 650 amino acid residues. In a particular embodiment, in order to facilitate the detection, a FLAG tag was inserted into the carboxyl terminus of the polypeptide of the invention. This FLAG-tagged NOK amino acid sequence is shown in SEQ ID NO:8. SEQ ID NO:8 is consisted of 658 amino acid residues in which the FLAG tag is located from the 651st residue to 458th residue.

Further, the present invention provides an isolated polynucleotide which encodes a chimeric molecule formed between NOK and at least one heterogeneous polypeptide.

In a preferable embodiment, the present invention provides an isolated polynucleotide that encodes the chimeric receptor EPOR/NOK that is fused between the extracellular domain of mouse erythropoietin receptor and the transmembrane and intracellular domains of human NOK, wherein the polynucleotide comprises a nucleotide sequence selected from:
1) the nucleotide sequence of SEQ ID NO: 5
2) the nucleotide sequence encoding the amino acid sequence SEQ ID NO:6, and
3) a nucleotide sequence that has at least 90% identity with the nucleotide sequence of 1) or 2).

SEQ ID NO:5 is consisted of 1953 bases. The open reading frame of SEQ ID NO:5 is from the first nucleotide to 1953th nucleotide (5′ to 3′): the coding sequence for the extracellular domain of mouse EPOR starts from the first nucleotide to 750th nucleotide; a NotI restriction endonuclease recognition site is located at 751st to 758th nucleotide; the transmembrane and intracellular domains of NOK is from the nucleotide 759th to nucleotide 1950th.

In a particular embodiment, in order to facilitate the detection, a sequence encoding a FLAG tag was added to the 3′ end of the polynucleotide of the invention. The DNA sequence that encodes the FLAG-tagged NOK gene is shown in SEQ ID NO:7. SEQ ID NO:7 is consisted of 1977 nucleotides in which the FLAG tag is located from the nucleotide 1951st to 1977th.

In a further aspect, the present invention provides a vector comprising a polynucleotide encoding a chimeric receptor EPOR/NOK which is a fusion formed between the extracellular domain of the mouse erythropoietin receptor and the transmembrane and intracellular domains of human NOK. In a particular embodiment, the vector of the invention comprising a polynucleotide encoding the chimeric receptor EPOR/NOK is pcDNA3(EPOR/NOK).

The present invention also provides a host cell transformed with a vector comprising a polynucleotide encoding the chimeric receptor EPOR/NOK which is a fusion formed between the extracellular domain of the mouse erythropoietin receptor and the transmembrane and intracellular domains of human NOK. In a particular embodiment, the host cell of the present invention is the BaF3-EPOR NOK cell line that has been transformed with a vector comprising the polynucleotide encoding the chimeric receptor EPOR/NOK. This cell line was deposited in China General Microbiological Culture Collection Center (CGMCC) on May 9, 2004 with the deposit number of CGMCC No. 1144.

The inventors found that stable expression of EPOR/NOK gene in BaF3 cells resulted in transformation of the BaF3 cells, and the growth of BaF3 cells changed from an IL-3 dependent pattern to an IL-3 independent pattern. Injection (s.c.) of BaF3-EPOR/NOK cells stably expressing EPOR/NOK into nude mice resulted in not only tumor formation at the injection site but also metastasis at multiple distant organs, which is the typical manifestations of malignant tumor. BaF3-EPOR/NOK injected nude mice can serve as a model system for the study of the mechanisms of tumorigenesis and metastasis, and for screening an agent with anti-tumorigenesis and anti-metastasis activities. In addition, BaF3-EPOR/NOK cell also provides a good cellular model system for screening or evaluating an anti-tumor agent against NOK-induced tumorigenesis and metastasis.

In yet another aspect, the present invention also provides a method of preparing the protein or the fusion protein of the invention. The method comprises the steps of culturing a host cell comprising a polynucleotide encoding the NOK protein or its fusion protein under a condition suitable for the expression of the NOK or its fusion protein, and collecting the expression product. The methods and conditions that can be used in the invention for cell culture and protein purification are well recognized by a person skilled in the art.

The polypeptide of the invention can be produced in the following ways, such as, but not limited to, by purification from native polypeptide, expression of recombinant polypeptide, and chemical synthesis. For example, the cells that are capable of expressing the polypeptide of the invention are cultured under culture conditions in which the polypeptide of the invention can be effectively produced. Such effective culture conditions include (but not limited to) the following conditions such as the effective culture medium, biological reactor, temperature, pH, and oxygen. The effective culture medium represents any medium that can support the growth of the cells for the production of the polypeptide of the invention. The culture conditions are well known for those skilled in the art.

In a further aspect, the present invention also provides an antibody that specifically binds to the NOK protein of the invention. The antibody can be obtained by immunizing an animal with a putative epitope of the polypeptide, the sequence of which corresponds to the 360th to 380th amino acid residues of NOK protein, as shown in SEQ ID NO:10. The present invention also provides the nucleic acid encoding the above putative epitope, which has a nucleotide sequence as shown in SEQ ID NO:9 that corresponds to a 61-nucleotide region of NOK coding sequence from the 1078th base to 1140th base. The antibody can be either polyclonal or monoclonal. The antibody of the invention may be a chimeric, single-chained, and humanized antibody, or a Fab fragment or the product of Fab expression library. The method used for producing these antibodies and fragments are well known in the art.

It is found that NOK gene is over-expressed in many tumor tissues such as head and neck cancers, gastroenteric cancers, and skin cancers, as shown by tumor microarray analysis using the polyclonal antibody produced with the putative epitope of NOK polypeptide.

In another aspect, the present invention also provides oligonucleotide probes or primers that can hybridize with the polynucleotide of the invention.

The oligonucleotide probes or primers of the invention can be RNA, DNA or the derivatives of the RNA or DNA. The minimal length of this type of oligonucleotide is a length that is required to form a stable hybrid between the oligonucleotide and its complementary sequence in the nucleic acid molecule of the invention. The oligonucleotides can selectively hybridize with the polynucleotide of the invention under high stringent condition. By “high stringent condition”, it refers to (1) washing condition that is conducted at low ion strength and/or high temperature, for example, 0.015M NaCl/0.0015M sodium citrate/0.1% NaDodSO₄, at 50° C.; (2) use of the denature reagent formamide during hybridization, for example, 50% (vol/vol) of formamide and 0.1% bovine serum albumin (BSA), 0.1% Ficoll, 0.1% polyvinylpyrrolidone (PVP), 50 mM phosphate buffer, pH7.5, and 750 mM NaCl, 75 mM sodium citrate, at 42° C.; or (3) use of 50% of formamide, 5×SSC (0.75M NaCl, 0.075M sodium citrate), 5 mM Na₃PO₄ (pH 6.8), 0.1% Sodium pyrophosphate, 5×Denhardt's solution, salmon sperm DNA (50 mg/ml), 0.1% SDS and 10% dextran sulfate, at 42° C., dissolved in 0.2×SSC and 0.1% SDS.

The oligonucleotide probes or primers or the antibody of the invention can be used in the diagnosis of disease associated with the polynucleotide, polypeptide of the invention or the mutant derivatives thereof in a subject or the susceptibility of a subject to said disease.

The inventors discovered that a single mutation at tyrosine 327 or 356 (tyrosine→phenylalanine) of NOK can not only prevent the multiple mitogenic signaling pathways, but also inhibit colony formation of the mutated stable cells. Furthermore, the inventors also provide the single point mutant form of EPOR/NOK at either tyrosine 327 or 356 (tyrosine→phenylalanine) of NOK. Both mutants can effectively abolish the NOK-induced tumorigenesis as well as prevent multiple mitogenic and metastasis-related signaling pathways.

The present invention also provides a NOK transgenic animal model. The inventors discovered that over-expression of NOK in transgenic mice induced B cell lymphoma/leukemia like disease. Thus, NOK transgenic mice could serve as a useful model system to study the formation of B cell lymphoma/leukemia and to screen potential therapeutic agents for such diseases.

The present invention also demonstrated that NOK gene was over-expressed in head and neck cancers, suggesting that NOK may serve as the potential diagnostic marker for head and neck cancers, and may also be the target for screening and developing therapeutic agents against the related diseases.

The inventors also discovered that NOK gene was over-expressed in gastroenteric (such as colon and rectum) cancers, thyroid carcinoma, and skin cancer. Thus, NOK may serve as the potential therapeutic target and/or clinical diagnostic marker for these diseases.

The following non-limiting examples are exemplary embodiments of the invention. As can be understood by those skilled in the art, many modifications to the exemplary embodiments described herein are possible. The invention is intended to encompass all such modifications within its scope.

### EXAMPLES

Unless otherwise specified, the recombinant DNA techniques used in the following experiments are standard techniques well known by those skilled in the art. Such techniques are described for example in J. Perbal's “A Practical Guide to Molecular Cloning” (John Wiley and Sons (1984)), J. Sambrook et al “Molecular Cloning: A Laboratory Manual” (Cold Spring Harbour Laboratory Press (1989)), T. A. Brown (ed) “Essential Molecular Biology: A Practical Approach” (IRL Press (1991)), and D. M. Glover and B. D. Hames (ed) “DNA Cloning: A Practical Approach” (IRL Press (1995 and 1996)).

### Example 1

### Cloning of NOK Gene and Construction of the Plasmid pcDNA3-NOK

The full length cDNA of human NOK was obtained by RT-PCR from total RNAs prepared from excised human amygdala tumor tissue (provided by the Surgical Department of Peking Union Medical College Hospital). Total RNAs were isolated by using RNAzol extract kit (Life Technologies). RT-PCR was conducted using the one step RT-PCR kit (Takara) following the manufacturer's instruction, with the following primers:

5′-TATAAAGCTTATGGGCATGATGACACGGATGCT-3′(SEQ ID NO: 11) and

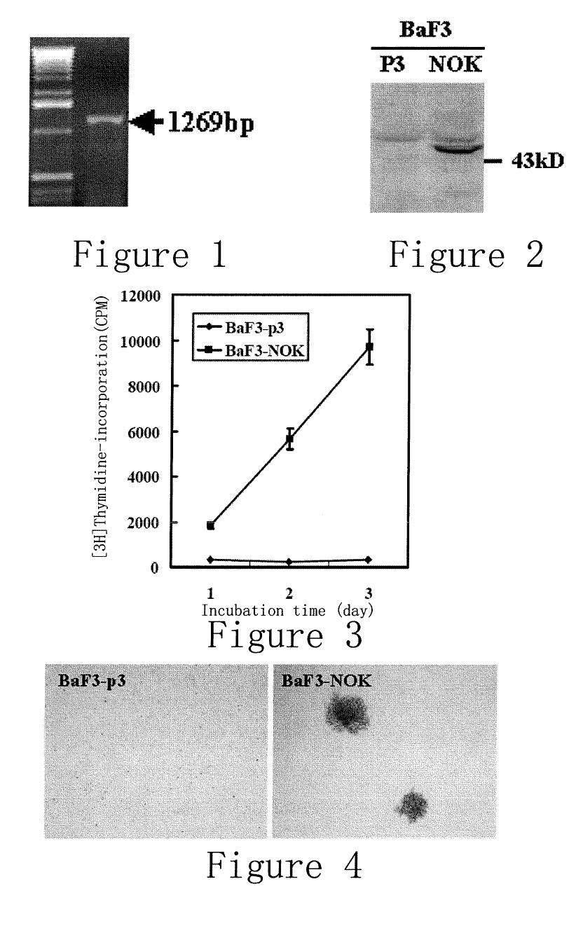

5′-TATACTCGAGTCAGGCGTAGTCGGGCACGTCGTAGGGGTAAAGCATGC TATAGTTGTA-3′(SEQ ID NO:12), respectively. (The underlined represents the HA coding sequence). The PCR products (FIG. 1) were purified and then subcloned into pGEM-T vector (Promega) and confirmed by restriction endonuclease digestion and DNA sequencing. The sequenced RT-PCR product was subcloned into the HindIII and XhoI sites of pCDNA3 (invitrogen) to form pCDNA3-NOK. DNA sequencing demonstrated that NOK gene has the nucleotide sequence of SEQ ID NO:1. The fusion gene with HA tag (SEQ ID NO:3) can be recognized by mouse anti-HA monoclonal antibody.

The protein encoded by NOK gene has the amino acid sequence of SEQ ID NO:2 and comprises a typical tyrosine kinase domain (amino acid residues from 105 to 327).

### Example 2

### Establishment of BaF3-NOK Stable Cell Line

About 1×10⁶ BaF3 cells (a murine pre-B cell line purchased from Cell Center of Institute of Biochemistry and Cell Biology (Shanghai), Chinese Academy of Sciences) were collected by centrifugation, and then resuspended in 0.5 ml serum free RPMI-1640 medium (Gibco). About 3 μg of pcDNA3-NOK from example 1 was mixed with BaF3 cells. After 10 minutes incubation at 4° C., the mixed cells were electroporated using a ECM399 Electroporator (BTX) at 1500 uF and 220-230 V (t˜25-35 msec). Transfected cells were first plated on 96-well plate and selected in the presence of 1000 μg/ml of G418 for 10 days. Then, selected resistant clones were expanded in 10 cm culture dishes for further analysis. The G418 resistant clone BaF3-NOK has the deposit number CGMCC No. 1145.

### Example 3

### Detection of NOK Gene Expression in BaF3-NOK Cells

Since NOK-HA (SEQ ID NO:4) has a HA tag at its carboxyl terminus, the positive clone can be detected by western blot analysis. Equal amount of cell lysates from both BaF3-NOK and BaF3-p3 control (BaF3 cell stably transfected with the empty vector pcDNA3) cells were loaded onto 10% SDS-PAGE. After separation, the reaction product was transferred to nitrocellulose membrane (Amersham Biosciences). Hybridization was conducted by using mouse anti-HA monoclonal antibody (Santa Cruzs), followed by horseradish peroxidase-conjugated secondary antibodies, and developed by using enhanced chemiluminescence (ECL) according to the manual description (Amersham Biosciences). The result shown in FIG. 2 clearly demonstrated that BaF3-NOK could express the NOK protein with a molecule weight around 45 kD.

### Example 4

### IL-3 Independent Growth of BaF3-NOK Stable Cells

BaF3 cell are murine pre-B cell line. The growth of this type of cell is dependent on the presence of interleukin-3 in culture medium. Stable BaF3 cells (1×10⁵) grew at a starvation condition without WEHI-3B (purchased from Cell Center of Institute of Biochemistry and Cell Biology, Chinese Academy of Sciences) condition medium (IL-3) for 3 consecutive days in RPMI-1640 medium plus 1% fetal calf serum (FBS), and each well was mixed with one micro curie [³H] thymidine at 5 hours before harvest. The result presented in FIG. 3 demonstrated that in the absence of IL-3, BaF3-NOK could proliferate for at least 3 days, whereas the control cell line BaF3-p3 was unable to proliferate in the absence of IL-3 stimulation, indicating that BaF3-NOK can proliferate in the absence of WEHI-3B conditional medium (without IL-3).

### Example 5

### Anchorage-Independent Growth of BaF3-NOK Cells Under Starvation Condition (Low Serum and Free of IL-3)

The detection of colony formation in soft agar is an important index to evaluate the transformation potential of a stable cell line. About 1×10⁵ stable BaF3 were resuspended in 5 ml of 0.4% top agar in RPMI-1640 supplemented with 1% FBS and 400 μg/ml G418 which was layered over a 5 ml of 0.8% bottom agar dissolved in RPMI-1640 with 1% FBS plus 400 μg/ml G418 in a 60 mm culture dish. After three weeks, the anchorage-independent colonies were visualized with Nikon microscopy, and the colony diameter more than 0.1 mm was regarded as positive. The results shown in FIG. 4 indicate that BaF3-NOK could induce colony formation in a starvation condition. Statistical analysis in Table 1 demonstrates that the stable expression of NOK gene in BaF3 cells can transform this cell line from growth factor dependent growth to the tumor growth characters.

TABLE 1
Transformation assay-anchorage independent
growth by using BaF3 stable cells

| BaF3 | Cell number | Colony number (SD)* |
|---|---|---|
| BaF3-p3 | 1 × 10⁵ | 0 (0) |
| BaF3-NOK | 1 × 10⁵ | 206 (18) |

*Results represent the mean ± SD of three independent experiments.

### Example 6

### Induction of Tumorigenesis and Metastasis after s.c. Injection of BaF3-NOK into Nude Mice

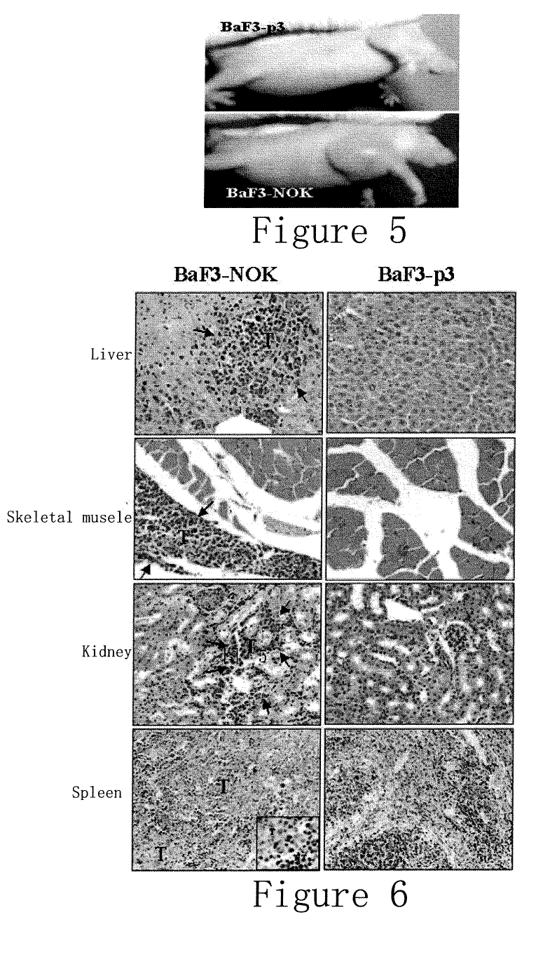

Four to six-week old athymic, BALB/c nude mice were subcutaneously injected with BaF3 control (wild type stably transfected with empty vector pcDNA3.0) or stable cells expressing NOK with cell number≈1.0×10⁷ into the right superior flanks. Each group had six mice including 3 males and 3 females. Tumor formation could be observed after one week injection with BaF3-NOK at the injection site. FIG. 5 shows tumor formation in both experimental and control groups after 2 week inoculation. Three weeks later, the mice started to development of cachexia such as loss weights and slow moving, and usually died within 30 days. The stable BaF3-NOK cells behaved like a malignant tumor which did not have an envelope and could actively grow and penetrate into the adjacent skeletal muscle underneath and massively distributed within the inter-fiber compartments (FIG. 6). The metastatic tumor cells BaF3-NOK cells were prevalent in mouse liver, spleen, kidney, and skeletal muscle etc. Table 2 shows that the average weight of liver in BaF3-NOK inoculated mice increased 2.4 folds (3.09±0.62 g versus 1.30±0.25 g), whereas the average weight of spleen in BaF3-NOK mice increased even more severely to about 8.7 folds (0.78±0.20 g versus 0.09±0.02 g). In liver, the infiltration of tumor cells disrupted the plate arrangement of hepatocytes in the lobules (FIG. 6). Under higher magnification, abnormal mitotic figures could be clearly identified in liver section (FIG. 6). In kidney, the tumor cells were penetrated through arcuate vein, and then infiltrated and spread into the interspace of renal columns, implying that the spreading of tumor cells to distant organs might be directly through blood vessels (FIG. 6). Although spleen is an unusual organ for tumor metastasis, these NOK expressing cells frequently promoted the dissemination of transformed cells into spleen. These observations may suggest the preferential dissemination and/or the aggressive character of these tumor cells in vivo.

TABLE 2
Tumor formation in nude mice*

| Cell line | Sex | Cell number | Time (day) | Name | Tumor (g) | Liver (g) | Spleen (g) |
|---|---|---|---|---|---|---|---|
| BaF3-p3 | M | 1.0 × 10⁷ | 28 | M-1 | — | 1.66 | 0.10 |
| | | | | M-2 | — | 1.37 | 0.12 |
| | | | | M-3 | — | 1.45 | 0.08 |
| | F | 1.0 × 10⁷ | 28 | F-1 | — | 1.24 | 0.10 |
| | | | | F-2 | — | 0.95 | 0.05 |
| | | | | F-3 | — | 1.11 | 0.08 |
| | | | | Average^{a} | — | 1.30 ± 0.25 | 0.09 ± 0.02 |
| BaF3-NOK | M | 1.0 × 10⁷ | 28 | M-1 | 5.24 | 3.02 | 0.65 |
| | | | | M-2 | 4.67 | 4.20 | 1.06 |
| | | | | M-3 | 5.41 | 3.17 | 0.83 |
| | F | 1.0 × 10⁷ | 28 | F-1 | 6.44 | 3.17 | 0.94 |
| | | | | F-2 | 4.18 | 2.52 | 0.57 |
| | | | | F-3 | 4.59 | 2.48 | 0.61 |
| | | | | Average^{a} | 5.09 ± 0.80 | 3.09 ± 0.62 | 0.78 ± 0.20 |

*Results represent the mean ± SD of three independent experiments

### Example 7

### Construction of Chimeric Receptor EPOR/NOK

In order to understand the function of the polypeptide of the invention, the inventors created the chimeric polypeptide EPOR/NOK that was fused between the extracellular domain of mouse erythropoietin receptor (EPOR) and the transmembrane and intracellular domains of human NOK, and studied the expression and function of this fusion gene.

The interaction between erythropoietin (EPO) and erythropoietin receptor (EPOR) play a very important role during the growth and differentiation of bone marrow erythroid progenitor cells (Heath D S et al. Separation of the erythropoietin-responsive progenitors BFU-E and CFU-E in mouse bone marrow by unit gravity sedimentation. Blood 47:777-792, 1976). EPOR is a typical member of type I cytokine receptor superfamily. The extracellular part of this type of receptor usually contains four cysteine residues at the N terminus and a Trp-Ser-X-Trp-Ser (WSXWS) motif at the C-terminus closed to transmembrane domain. The WSXWS motif is important for ligand-receptor recognition. Box 1 is an intracellular motif proximal to the transmembrane helix, and is usually composed of a conserved Pro-Xaa-Pro motif proceeding with a cluster of five hydrophobic amino acid residues, while box 2 is less conserved and is often located at the distant region to the transmembrane domain (Jiang N, et al. The box1 domain of the erythropoietin receptor specifies Janus kinase 2 activation and functions mitogenically within an interleukin 2 beta-receptor chimera. J Biol Chem. 1996 Jul. 12; 271(28):16472-6). The activation of EPOR is dependent on the receptor dimerization that in turn to stimulate the adaptor protein JAK2 and to phosphorylate the transcriptional factors such as STAT5 for activating downstream targeting gene expressions (Klingmuller U, et al. Multiple tyrosine residues in the cytosolic domain of the erythropoietin receptor promote activation of STAT5. Proc Natl Acad Sci USA. 1996 Aug. 6; 93(16):8324-8; Barber D L, et al. A common epitope is shared by activated signal transducer and activator of transcription-5 (STAT5) and the phosphorylated erythropoietin receptor: implications for the docking model of STAT activation. Blood. 2001 Apr. 15; 97(8):2230-7). To facilitate the functional study on novel type I cytokine receptor, the extracellular domain of EPOR is usually fused with the transmembrane and intracellular domain of a novel receptor. The advantage of this type of chimeric receptor is that the activation of intracellular signaling pathways can be studied even in the absence of specific ligand.

In order to obtain the EPOR/NOK, the extracellular domain of EPOR was first isolated by using the primers:

5′-TATAGCGATATCATGGACAAACTCAGGGTGCC-3′ (SEQ ID NO:13) and

5′-TATAGCGCGGCCGCGAGAGGGTCCAGGTCGCTA-3′ (SEQ ID NO:14)

The plasmid pMX-EPOR(pBabe-EPO-R) (PNAS, Vol. 93, p8324-8328, August 1996) was served as template for PCR. The PCR reaction contained followings: 50 ng template DNA □100 pmol of each primer □1× reaction buffer □200 μmol/1 for each dNTP □1 unit of Taq DNA polymerase with high fidelity (Takara) with the cycle of 94° C./5 min, 94° C./30 sec, 55° C./30 sec, and 72° C./1 min for 35 cycles □ and finally extended at 72° C. for 10 min. The amplified extracellular domain of EPOR was subcloned into the EcoRV and NotI sites of pcDNA3 (Invitrogen) to generate the plasmid pcDNA3(EPOR).

The following primers:

(SEQ ID NO:15)

5′-TATAGCGGCCGCAGTGATTATCGTCCCAACTTT-3′
and

(SEQ ID NO:16)

5′-TATACCAGTGTGCTGGTCACTTGTCATCGTCGTCCTTGTAGTCAAGC
ATGCTATAGTTGTAGA-3′

were used to amplify the transmembrane and intracellular domain of NOK by using pcDNA3-NOK as a template. The PCR reaction contained followings: 50 ng template DNA □100 pmol of each primer □1× reaction buffer □200 μmol/1 for each dNTP □1 unit of Taq DNA polymerase with high fidelity (Takara) with the cycle of 94° C./5 min, 94° C./30 sec, 55° C./30 sec, and 72° C./1 min for 35 cycles □ and finally extended at 72° C. for 10 min. The amplified transmembrane and intracellular domain of NOK was subcloned into the NotI and BstXI sites of pcDNA3 (Invitrogen) to generate the plasmid pcDNA3-EPOR/NOK. This chimeric receptor with a FLAG tag has the nucleotide sequence of SEQ ID NO:7 that can encode the amino acid sequence of SEQ ID NO:8. The chimeric receptor can be recognized by using anti-M2 antibody.

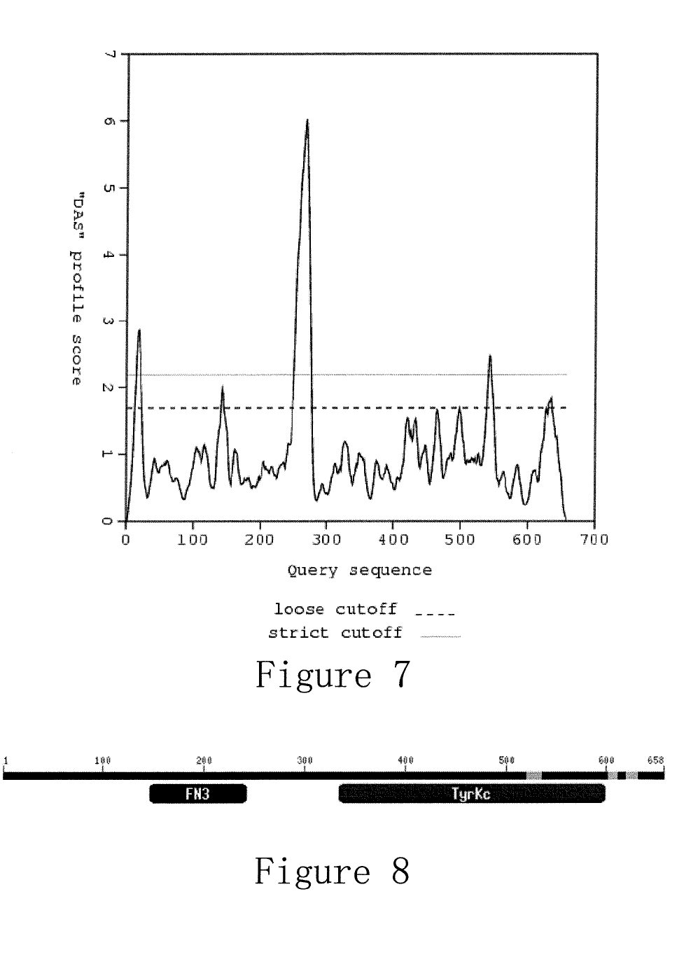

EPOR/NOK protein has the amino acid sequence of SEQ ID NO: 6. DAS program analysis indicates that EPOR/NOK is a receptor molecule with a single transmembrane helix which is located from 249th amino acid residue to 277th amino acid residue as shown in FIG. 7. EPOR/NOK protein has a typical tyrosine kinase domain (from 333rd to 600th amino acid residue) as shown in FIG. 8

### Example 8

### Establishment of BaF3-EPOR/NOK Stable Cell Line

About 1×10⁶ BaF3 cells (a murine pre-B cell line purchased from Cell Center of Institute of Biochemistry and Cell Biology, Chinese Academy of Sciences) were collected by centrifugation, and then resuspended into 0.5 ml serum free RPMI-1640 medium (Gibco). About 3 μg of pcDNA3-EPOR/NOK from example 7 was mixed with BaF3 cells. After 10 minutes incubation at 4° C., the mixed cells were electroporated using a BTX machine at 1500 uF and 220-230 V (t˜25-35 msec). Transfected cells were first plated on 96-well plate and selected in the presence of 1000 μg/ml of G418 for 10 days. Then, selected resistant clones were expanded in 10 cm culture dishes for further analysis. The G418 resistant clone BaF3-EPOR/NOK has the deposit number CGMCC No. 1144.

### Example 9

### Detection of EPOR/NOK Expression in BaF3-EPOR/NOK Cells

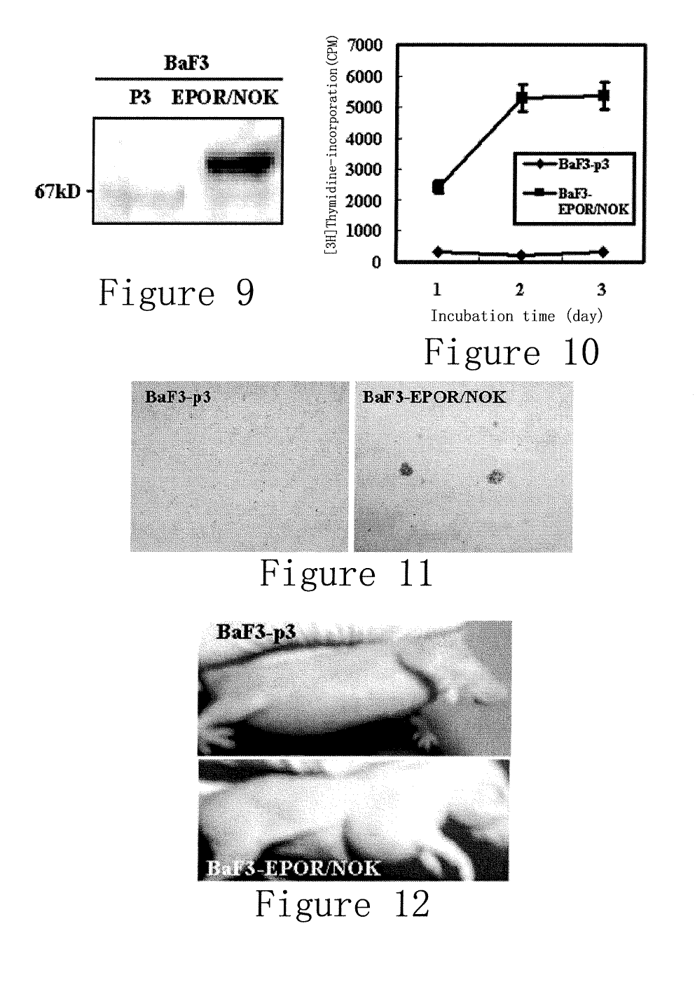

Since EPOR/NOK (SEQ ID NO: 7) has a FLAG tag at its carboxyl terminus, the positive clone can be detected by western blot analysis. Equal amount of cell lysates from both BaF3-EPOR/NOK and BaF3-p3 control (BaF3 cell stably transfected with the empty vector pcDNA3) cells were loaded onto 10% SDS-PAGE. After separation, the reaction product was transferred to nitrocellulose membrane (Amersham Biosciences). Hybridization was conducted by using mouse anti-FLAG monoclonal antibody (Santa Cruzs), followed by horseradish peroxidase-conjugated secondary antibodies, and developed by using enhanced chemiluminescence (ECL) according to the manual description (Amersham Biosciences). The result shown in FIG. 9 clearly demonstrated that BaF3-EPOR/NOK could express the EPOR/NOK protein with a molecule weight around 68 kD.

### Example 10

### IL-3 Independent Growth of EPOR/NOK Transformed BaF3 Cells

BaF3 cell are murine pre-B cell line. The growth of this type of cell is dependent on the presence of interleukin-3. Stable BaF3-EPOR/NOK cells (1×10⁵) grew at a starvation condition without WEHI-3B (purchased from Cell Center of Institute of Biochemistry and Cell Biology, Chinese Academy of Sciences) conditional medium (IL-3) for 3 consecutive days in RPMI-1640 medium plus 1% fetal calf serum (FBS), and each well was mixed with one micro curie [³H] thymidine at 5 hours before harvest. The result presented in FIG. 10 demonstrates that in the absence of IL-3, BaF3-EPOR/NOK could proliferate for at least 3 days, whereas the control cell line BaF3-p3 was unable to proliferate in the absence of IL-3 stimulation, indicating that BaF3-EPOR/NOK can proliferate in the absence of WEHI-3B condition medium (provided with IL-3).

### Example 11

### Anchorage-Independent Growth of BaF3-EPOR/NOK Under Starvation Condition (Low Serum and Free of IL-3)

The detection of colony formation in soft agar is an important index to evaluate the transformation ability of a stable cell line. About 1×10⁵ stable BaF3-EPOR/NOK cells were resuspended in 5 ml of 0.4% top agar in RPMI-1640 supplemented with 1% FBS and 400 μg/ml G418 which was layered over a 5 ml of 0.8% bottom agar dissolved in RPMI-1640 with 1% FBS plus 400 μg/ml G418 in a 60 mm culture dish. After three weeks, the anchorage-independent colonies were visualized with Nikon microscopy, and the colony diameter more than 0.1 mm was regarded as positive. The results shown in FIG. 11 indicate that BaF3-EPOR/NOK could induce colony formation in a starvation condition. Statistical analysis in Table 3 demonstrates that the stable expression of EPOR/NOK gene in BaF3 cells can transform this cell line from growth factor dependent growth to growth factor independent growth.

TABLE 3
Transformation assay-anchorage independent
growth by using BaF3 stable cells

| BaF3 cell line | Cell number | Colony number(SD)* |
|---|---|---|
| BaF3-p3 | 1 × 10⁵ | 0 (0) |
| BaF3-EPOR/NOK | 1 × 10⁵ | 102 (10) |

*Results represent the mean ± SD of three independent experiments.

### Example 12

### Induction of Tumorigenesis and Metastasis after s.c. Injection of BaF3-EPOR/NOK Into Nude Mice

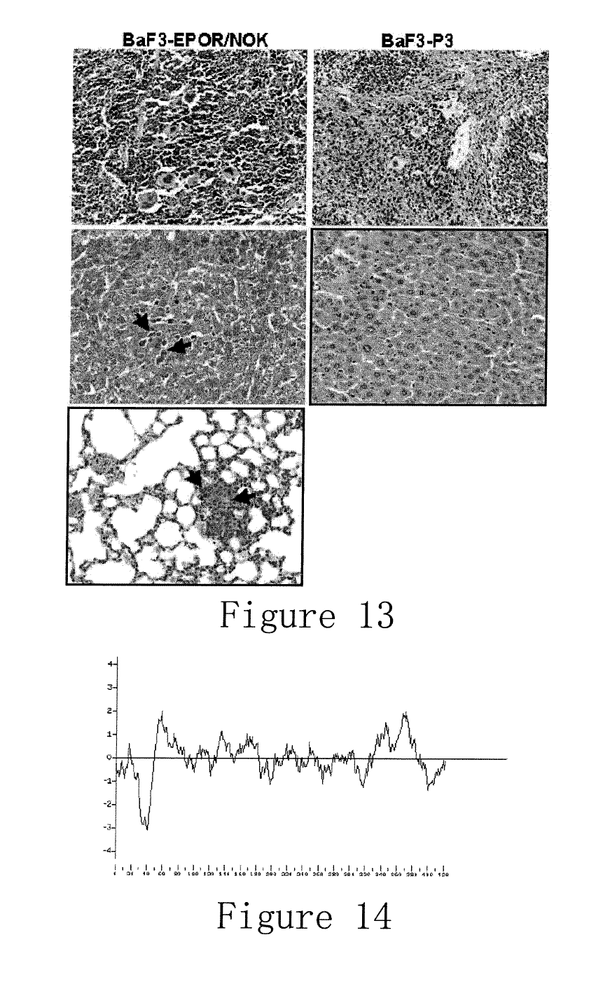

Four to six-week old athymic, BALB/c nude mice were subcutaneously injected with BaF3 control (wild type stably transfected with empty vector pcDNA3.0) or stable cells expressing NOK with cell number≈1.0×10⁷ into the right superior flanks. Each group had six mice including 3 males and 3 females. Tumor formation could be observed after one week injection with BaF3-NOK at the injection site. FIG. 12 shows tumor formation in both experimental and control groups after 3-week inoculation. The tumor formation induced by BaF3-EPOR/NOK had a less aggressive character than that of BaF3-NOK. Four weeks later, the mice started to development of cachexia such as loss weights and slow moving, and usually died within 35-40 days. The metastatic tumor cells BaF3-EPOR/NOK cells were found prevalently in mouse liver, spleen, kidney, and skeletal muscle etc. Table 4 shows that the average weight of liver in BaF3-EPOR/NOK inoculated mice increased from 1.3±0.25 g to 1.77±0.59 g, whereas the average weight of spleen in BaF3-EPOR/NOK mice increased even more significantly from 0.09±0.02 g to 0.20±0.08 g. BaF3-EPOR/NOK could induce malignant tumor formation in nude mice represented by the active growth of the tumor cells into the skeletal muscle underneath the injection site, the enlargements of liver and spleen, and tumor metastasis at multiple distant organs such as liver, spleen, kidney, and lung (FIG. 13 and Table 1).

TABLE 2
Tumor formation in nude mice*

| Cell line | Sex | Cell number | Time (Day) | Name | Tumor (g) | Liver (g) | Spleen (g) |
|---|---|---|---|---|---|---|---|
| BaF3-p3 | M | 1.0 × 10⁷ | 28 | M-1 | — | 1.66 | 0.10 |
| | | | | M-2 | — | 1.37 | 0.12 |
| | | | | M-3 | — | 1.45 | 0.08 |
| | F | 10 × 10⁷ | 28 | F-1 | — | 1.24 | 0.10 |
| | | | | F-2 | — | 0.95 | 0.05 |
| | | | | F-3 | — | 1.11 | 0.08 |
| | | | | Average^{a} | — | 1.30 ± 0.25 | 0.09 ± 0.02 |
| BaF3- | M | 1.0 × 10⁷ | 28 | M-1 | 1.81 | 2.00 | 0.20 |
| EPOR/NOK | | | | M-2 | 1.16 | 1.84 | 0.31 |
| | | | | M-3 | 2.55 | 2.79 | 0.27 |
| | F | 10 × 10⁷ | 28 | F-1 | 1.64 | 1.22 | 0.12 |
| | | | | F-2 | 1.20 | 1.21 | 0.10 |
| | | | | F-3 | 1.49 | 1.55 | 0.19 |
| | | | | Average^{a} | 1.64 ± 0.51 | 1.77 ± 0.59 | 0.20 ± 0.08 |

*Results represent the mean ± SD of three independent experiments.

### Example 13

### Prediction of the Epitope of NOK Protein and Generation of Specific Polyclonal Antibody

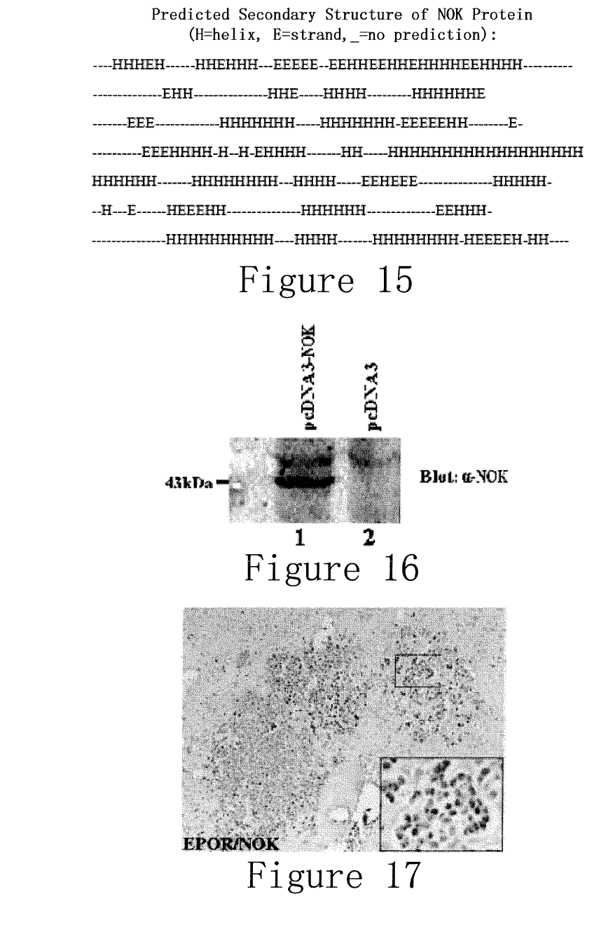

Analysis by using Kyte-Doolittle program (http://gcat.davidson.edu/rakamik/KD.html) on the hydropathy of NOK protein revealed that the fragments of amino acids 60-80 and 360-380 had the highest hydrophilicity as compared with the rests of the protein (FIG. 14). Protein secondary structure was analyzed by nnPredict (FIG. 15). 3D analysis by using 3D-PSSM Web Server V 2.6.0 (http://www.sbg.bio.ic.ac.uk/˜3dpssm/html/ffhome.html) revealed that the hydrophilic probability of the 360-380aa fragment was higher than that of the 60-80aa fragment. Therefore, SEQ ID NO: 9 was selected as the best epitope coding sequence, and SEQ ID NO:1 was served as a template to synthesize the NOK epitope for the production of NOK specific antibody. The 21 amino acid polypeptide (NOK epitope) was synthesized by SBS Genetech, Beijing. The synthesized epitopes were cross-linked with the maleimide-activated KLH (keyhole limpet hemocyanin) under the standard condition (PIERCE). 100 μg cross-linked epitopes (0.5 ml) were mixed with 0.5 ml of Freund's adjuvant complete and s.c. injected into a 2-kg New Zealand white rabbit (from the Animal Center of Peking University Medical School). The rabbit was boosted with 100 μg cross-linked epitopes mixed with Freund's adjuvant incomplete every three weeks for twice. After 6 weeks, the blood was taken from the carotid, and the separated serum was used for the analysis of NOK protein expression.

### Example 14

### Immunohistochemistical Detection of NOK Gene Expression Using Rabbit Anti-NOK Epitope Antibody

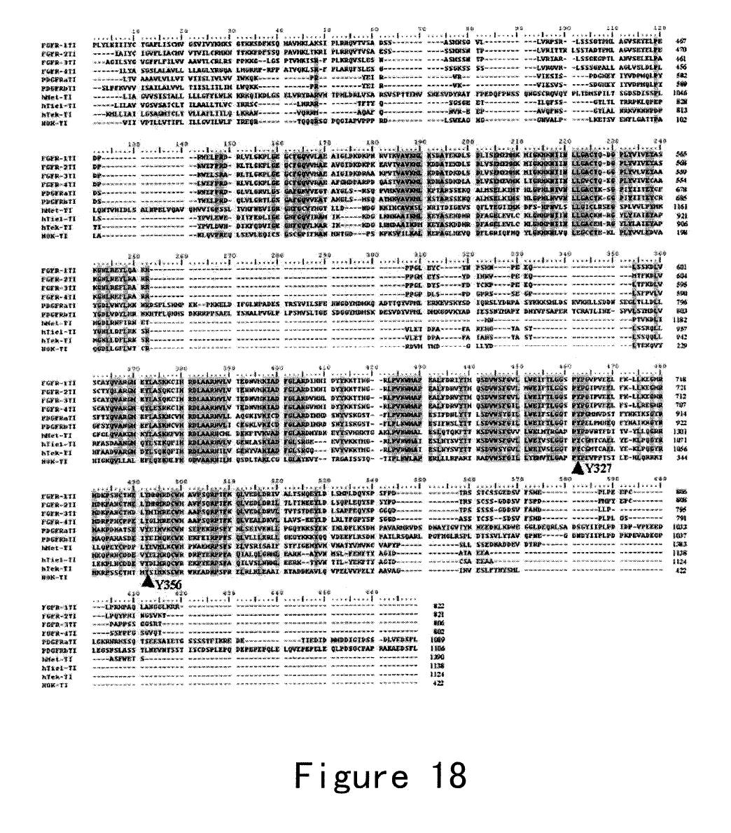

In order to detect the specificity of rabbit anti-NOK epitope antibody, the cell lysates were first harvested from the cells transiently transfected with pcDNA3.0-NOK and pcDNA3.0 (as a negative control), and then 15 μg of each protein sample was loaded and resolved onto 10% SDS-PAGE. The resolved reaction product was transferred to nitrocellulose membrane (Amersham Biosciences). Hybridization was conducted by using rabbit anti-NOK epitope antibody diluted at 1:4,000, followed by horseradish peroxidase-conjugated secondary antibodies, and developed by using enhanced chemiluminescence (ECL) according to the manual description (Amersham Biosciences). The result shown in FIG. 16 demonstrates that pcDNA3.0-NOK transfected cells could express the NOK protein with a molecule weight around 45 kD, and the predicted NOK epitope had a good antigenicity. In order to test whether anti-NOK epitope antibody could be good for immunohistochemistical analysis, the liver section prepared from BaF3-NOK injected nude mice was probed with either this primary antibody at a dilution of 1:800 or PBS as a negative control. FIG. 17 demonstrates the high level of NOK gene expression in the metastatic foci of liver.

### Example 15

### Role of Tyrosine Residues of Y327 and Y356 in NOK-Induced Tumorigenesis

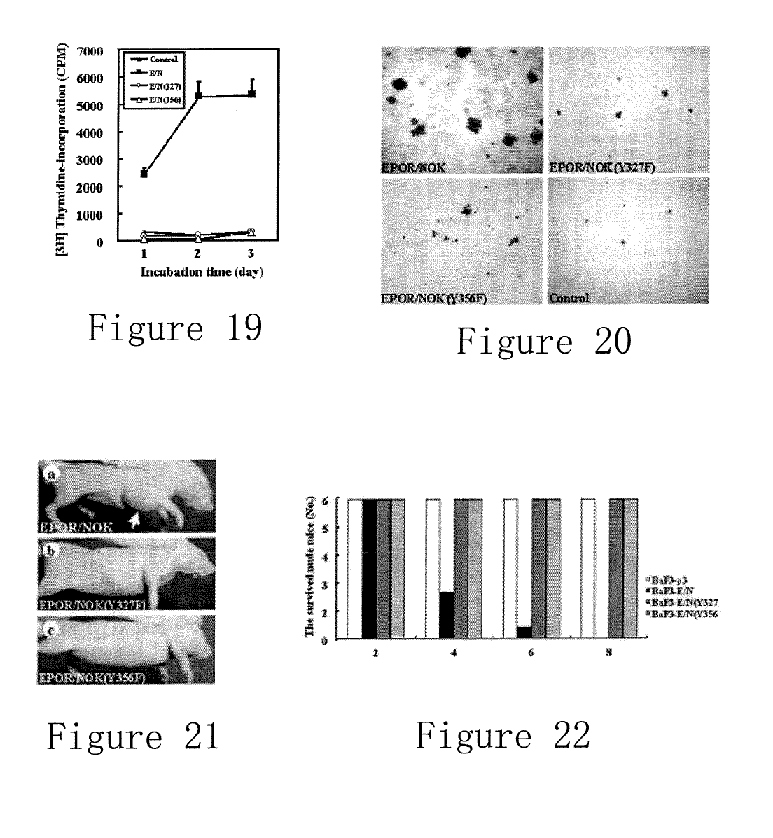

ClustalW (http://www.ebi.ac.uk/clustalw/) alignment was performed using transmembrane and intracellular domain isolated from FGFR1, FGFR2, FGFR3, FGFR4, PDGFRα, PDGFRβ, Met, Tie 1, Tek and NOK with GenBank accession numbers NP_{—}000595, CAA96492, P22607, AAB59389, P16234, P09619, AAA59591, P35590, NP_{—}000450 and AAT01226, respectively. Among them, two tyrosine sites of NOK protein, Tyr³²⁷ and Tyr³⁵⁶, were well conserved in all RPTKs examined. The mutant constructs of pcDNA3-EPOR/NOK(Y327F) and pcDNA3-EPOR/NOK(Y356F) were generated by Takara MutantBEST Kit (Takara Biotechnology Co., Ltd) by using pcDNA3-EPOR/NOK as a template as following the manual instruction. The mutant sense primers for Y327F and Y356F are 5′-cctcctaccagcatcctagagc-3′ (SEQ ID NO:17) and 5′-gcacacataccatgttcagtatcat-3′ (SEQ ID NO:18), respectively. The anti-sense PCR primers for Y327F and Y356F are 5′-gacttcaggaaacggtggtgct-3′ (SEQ ID NO:19) and 5′-agctactgggtctcttcatgatttt-3′ (SEQ ID NO:20), respectively. The reaction mixture was amplified by 30 cycles of PCR at the condition of 94° C. for 30 seconds, 55° C. for 30 seconds and 72° C. for 5 minutes. The PCR products were blunted at both ends and self-ligated with T4 DNA liagase. The mutant constructs were subsequently confirmed by sequencing analysis. BaF3-EPOR/NOK(Y327F) and BaF3-EPOR/NOK(Y356F) stable cells were generated by electroporating the wild type BaF3 cells with pcDNA3-EPOR/NOK(Y327F) and pcDNA3-EPOR/NOK(Y356F) as described in example 8. The proliferation potentials of these mutant stable cells were evaluated by [³H]thymidine incorporation assay at starvation condition (without WEHI-3B and serum). The replications of BaF3-EPOR/NOK(Y327F) and BaF3-EPOR/NOK(Y356F) stable cells were remained at basal levels as compared with that of the BaF3-EPOR/NOK stable cells during the 3-day incubation (FIG. 19). Single mutation at either Tyr³²⁷ or Tyr³⁵⁶ dramatically inhibited anchorage-independent growth (colony formation) of mutated BaF3 stable cells (FIG. 20).

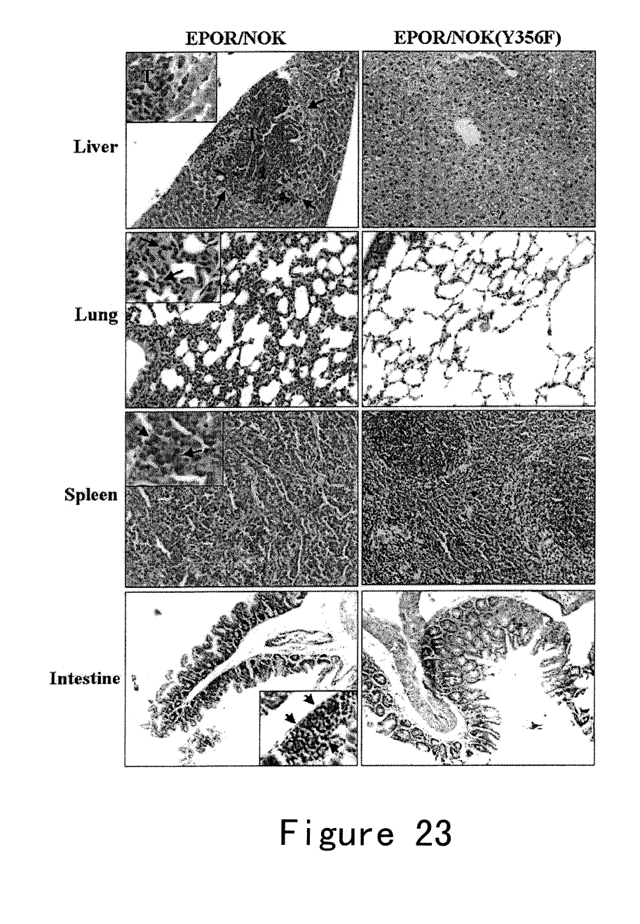

About 1×10⁷ stable cells from BaF3-p3, BaF3-EPOR/NOK, BaF3-EPOR/NOK(Y327F), or BaF3-EPOR/NOK(Y356F) were s.c injected into the right flanks of 4-6 week old nude mice. BaF3-EPOR/NOK injected mice appeared tumor growth at the injection sites after 4-week inoculation (FIG. 21). However, BaF3-E/N(Y327F) and BaF3-E/N(Y356F) mutant cells were completely unable to support tumor growth in nude mice for at least 4-5 months (Table 5). The survived numbers of BaF3-EPOR/NOK mice were dramatically reduced from 10 to 2.5 by 8^{th} week of inoculation (FIG. 22). The majority of BaF3-EPOR/NOK mice were deceased between 7 to 8 weeks of post-inoculation. In contrast, all mice receiving either BaF3-EPOR/NOK(Y327F) or BaF3-EPOR/NOK(Y356F) mutant cells were still in healthy state for at least 5 months, indicating that Tyr³²⁷ or Tyr³⁵⁶ site is crucial for NOK-induced tumorigenesis in nude mice. The spleen and liver were significantly enlarged in animals receiving BaF3-E/N inoculation with a mean value of 2.2% versus 0.58% (control) and 11.1% versus 5.5% (control), respectively, as compared with BaF3-P3 control (Table 5). However, no significant differences were observed in the spleens and livers from the animals injected with either BaF3-EPOR/NOK(Y327F) or BaF3-EPOR/NOK(Y356F) as compared with BaF3-P3 control (Table 5). BaF3-EPOR/NOK cells were also able to promote tumor metastasis at distant organs such as liver, lung, spleen, kidney, skeletal muscle and intestine (FIG. 23). Abrogation at either Tyr³²⁷ or Tyr³⁵⁶ residue was sufficiently to block the metastatic foci formation in nude mice.

TABLE 5
Analysis of nude mice injected with BaF3 stable cells and its mutant derivatives

| BaF3 | No. deceased/ | Body weight/ | Tumor weight/ | Liver weight/ | Spleen weight/ |
|---|---|---|---|---|---|
| stable cells | no. injected | median (g) | median (%)^{a} | median (%)^{b} | median (%)^{c} |
| P3 | 0/8 | 22.2-29.2/24.9 | 0/0 | 5.4-6.5/5.7 | 0.43-0.72/0.58 |
| E/N | 8/8 | 17.0-29.2/23.0 | 4.2-24.2/12.5 | 9.3-13.7/12.1 | 1.10-3.18/2.02 |
| E/N(Y327F) | 0/8 | 21.6-30.4/25.6 | 0/0 | 5.5-6.9/6.2 | 0.34-0.83/0.55 |
| E/N(Y356F) | 0/8 | 21.5-31.1/24.7 | 0/0 | 5.4-7.1/6.0 | 0.31-1.00/0.58 |

^{a, b,} and ^{c} represent the tumor, liver, or spleen weight divided by body weight for each animal, respectively

### Example 16

### Point Mutation at Either Tyrosine 327 or 356 Residue of NOK Attenuates Multiple Mitogenic Signaling Pathways

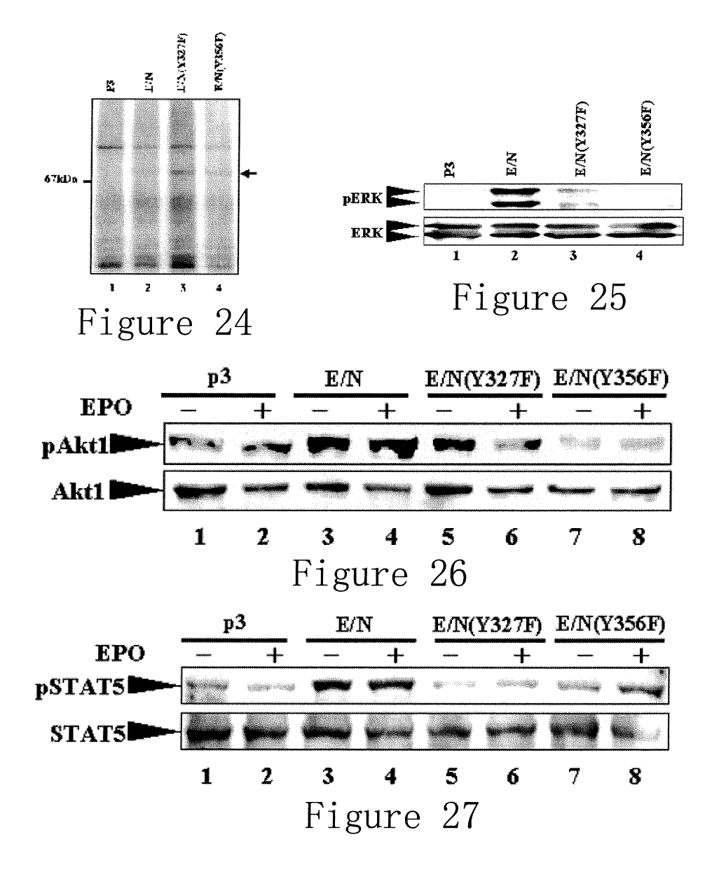

To directly address the mutagenetic effect of these point mutations (Y327F and Y356F) on NOK kinase activity, plasmid vectors carrying EPOR/NOK or its mutant derivatives were individually transfected into 293T cells. The cell lysates were immunoprecipitated with mouse anti-FLAG antibody. About 100 μCi of γ³²P-ATP was added to the reaction mixture for the detection of the kinase activities of NOK and its mutant derivatives. The result shows that point mutation at either Tyr³²⁷ or Tyr³⁵⁶ sites did not abolish their respective kinase activities, indicating that these two tyrosine sites are not functionally required for the kinase activity of NOK (FIG. 24). In order to detect the roles of these mutants in NOK mediated mitogenic signaling, cell lysates were prepared from BaF3-p3, BaF3-EPOR/NOK, BaF3-EPOR/NOK(Y327F) and BaF3-EPOR/NOK(Y356F) cells and subjected to western blot analysis. Mutation at Tyr³²⁷ (Y327F) severely reduced ERK activity, whereas Tyr³⁵⁶ mutation (Y356F) completely abolished ERK phosphorylation, indicating that Tyr³⁵⁶ residue is critical for the full activation of ERK pathway (FIG. 25). FIG. 26 demonstrates that, in the absence or presence of EPO, EPOR/NOK dramatically enhanced Akt phosphorylation, indicating that EPOR/NOK might be constitutively active and functioned in an EPO-independent manner, while both mutations significantly attenuated Akt activation with a more severe inhibition being seen in EPOR/NOK(Y356F) than that in EPOR/NOK(Y327F). Furthermore, examination on the phosphorylated STAT5 revealed that STAT5 could be activated by EPOR/NOK but not by EPOR/NOK(Y327F) or EPOR % NOK(Y356F) in the presence of 1% FBS, and this activation was independent of EPO stimulation (FIG. 27). Thus, single mutation at either Tyr³²⁷ or Tyr³⁵⁶ site was sufficiently to affect multiple downstream signaling pathways that might be critical for NOK-induced tumorigenesis.

### Example 17

### Over-Expression of NOK Attenuates Endogenous E-Cadherin Expression

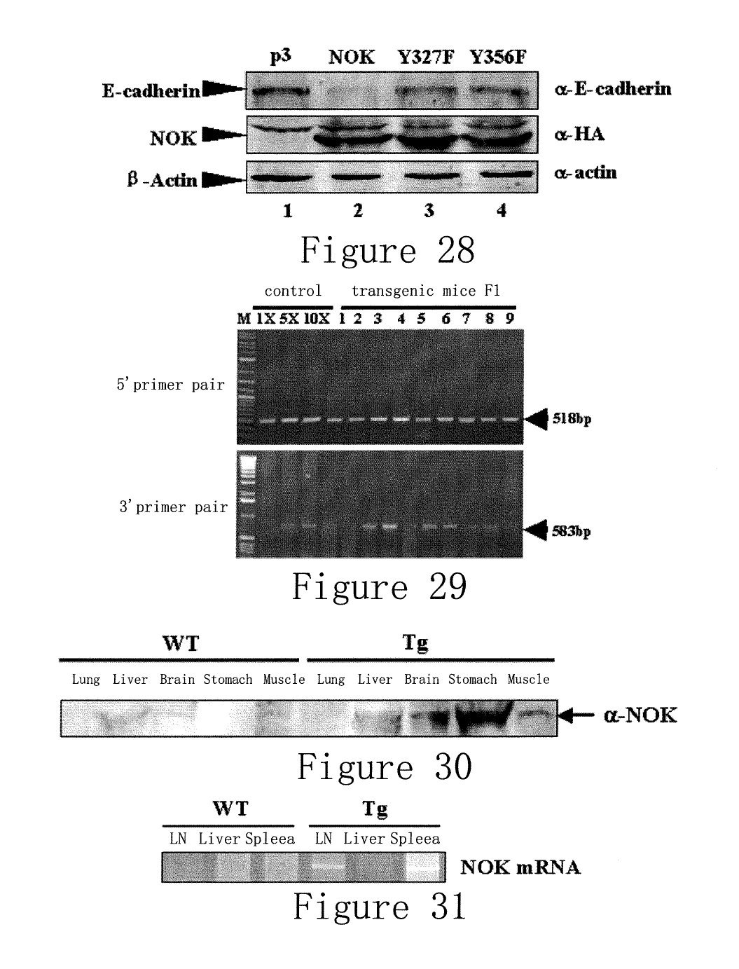

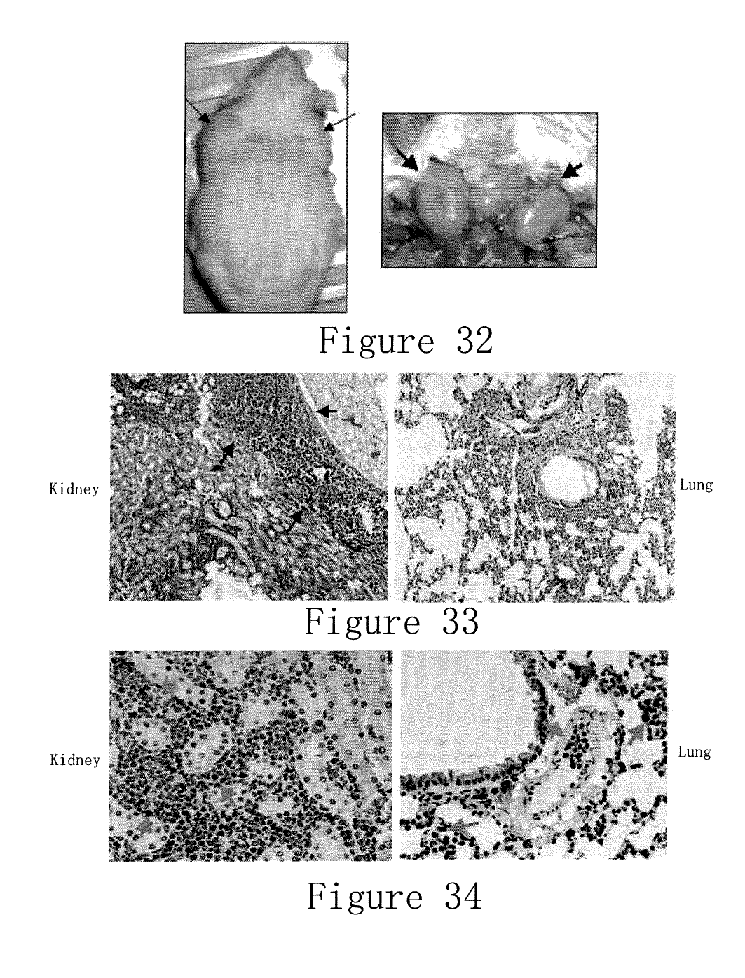

Studies indicate that down-regulation of E-cadherin in tumor cells can promote the migration and spread of the tumor cells, and causes tumor cell invasion and metastasis. To explore the possible role of E-cadherin in NOK-induced metastasis, 293T cells were transiently transfected with the HA-tagged wild type NOK (pcDNA3.0-NOK) and its two mutant derivatives [pcDNA3.0-NOK(Y327F) and pcDNA3.0-NOK(Y356F)]. Western blot shows that overexpression of NOK reduced endogeneous level of E-cadherin as compared with the P3 control (FIG. 28). However, single mutation at either Tyr³²⁷ or Tyr³⁵⁶ site did not significantly affect intracellular E-cadherin expression (FIG. 28). Thus, the result indicates that the metastatic effect of NOK could be at least partially induced by the down-regulation of E-cadherin expression in tumor cells.

### Example 18

### Establishment of NOK Transgenic Mice

Linearized NOK expression cassette was microinjected into the pronuclei of fertilized Kunming mouse oocytes and 20-25 fertilized oocytes were implanted into pseudopregnant female fosters to generate the transgenic mice. The transgenic founder mice were identified by genomic PCR, and then backcrossed with wild type Kunming mice, and the positive transgenic lines were maintained by inbreding between brothers and sisters. Genomic DNAs were extracted from the tails of transgenic mice. About 0.5 cm mouse tail was cut and incubated with 0.6 ml TNES lysis buffer (0.6% SDS, 0.4M NaCl, 0.1M EDTA, 0.01M Tris, pH7.5) plus 35 μl of proteinase K (10 mg/ml) at 56° C. overnight. After centrifugation, the supernatant was precipitated with two volumes of 100% ethanol. The DNA pellet was washed once with 70% ethanol before dissolving into sterile H2O. The primers for 5′ PCR product are:

(SEQ ID NO:21)

| | |
|---|---|
| CMV525-554: | 5′-tggcccgcctggcattatgcccagtacatg-3′, |
| and | |

(SEQ ID NO:22)

| | |
|---|---|
| FR4b111-140: | 5′-agccacaggatgaccccaagaaggatgagg-3′. |

The expected product is 518 bp;

The primers for 3′ PCR product are:

(SEQ ID NO:23)

| | |
|---|---|
| FR4b981-1010: | 5′-tcctgaagtccctcctaccagcatcctaga-3′, |
| and | |

(SEQ ID NO:24)

| | |
|---|---|
| BGH1210-1240: | 5′-tcttcccaatcctcccccttgctgtcctgc-3′. |

The expected product is 583 bp.

The PCR reaction was conducted as followings: 95° C. denatured for 5 min; amplified at 94° C./30 sec, 72° C./2 min for 35 cycles; the reaction product was finally extended at 72° C. for 10 min. The PCR result is shown in FIG. 29.

The expression profile of NOK gene in NOK transgenic mice was evaluated by both Western blot and RT-PCR analysis. Western blot analysis indicates that NOK protein could be detected in multiple tissues such as liver, brain, stomach, and skeletal muscle et al. as compared with the wild type control (FIG. 30). RT-PCR was conducted by using the primers:

5′-atgggcatgacacggatgct-3′ (SEQ ID NO:25) and

5′-tcaaagcatgctatagttg-3′ (SEQ ID NO:26) to amplify the full length of NOK cDNA.

The result shows that NOK mRNAs were present in the lymph node, liver, and spleen of the transgenic mouse (FIG. 31).

### Example 19

### Phenotypes and Pathogenic Changes of NOK Transgenic Mice

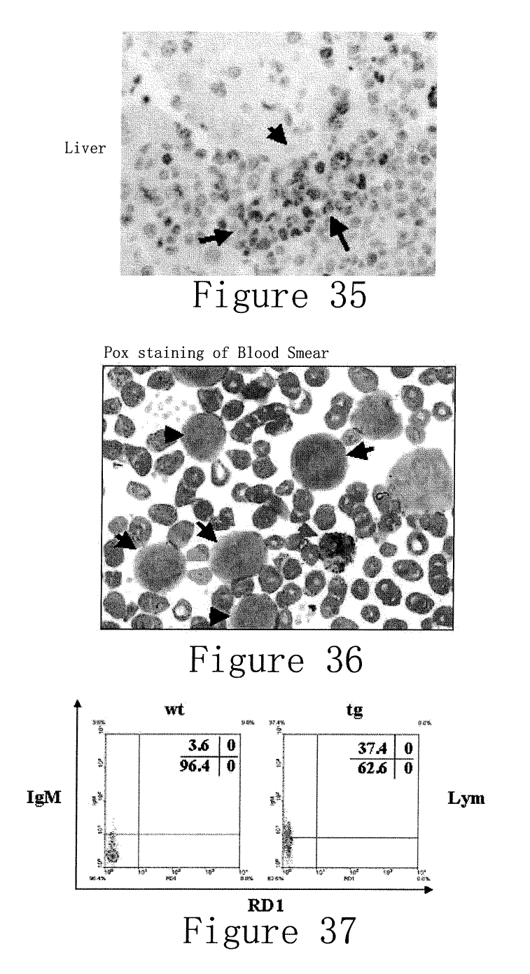

The main phenotypes of NOK transgenic mice include skin pruritus, abdominal distension, skin eschar, the enlargement of peripheral lymph nodes, and ankle joint swelling et al. In a non-SPF feeding and nursing condition, the life span of transgenic mice presented a seasonal death character. The numbers of animals died in spring and summer were usually higher than that died in fall and winter with a high peak at summer time. Sometime, the transgenic mice presented abnormal movement and even with muscle and limb cramps. Whole body anatomical analysis on more than 100 mice revealed that the pathogenesis of the major organs from different mice is heterogeneous. Many transgenic mice did not appear any visible abnormality in their major organs. In contrast, a significant group of NOK-transgenic mice presented the different degree of lymph node enlargement that can be most often found in cervicle, axillary, and abdominal lymph nodes. In many cases, the lung had flecked or diffused bleeding, or even consolidation at one or more lung lobes. In more severe cases, the mice presented the enlarged pulmonary portal lymph nodes and thymuses in the chest cavities, and had the color changes on the enlarged livers and spleens with patched and diffused metastatic foci across the entire organs. They also presented the enlarged mesenteric lymph nodes in their abdominal cavities. Although some mice did not have the enlarged livers and/or spleens, they could die from severe abdominal distension. The major organs of the transgenic mice such as liver, spleen, lymph node, kidney, stomach, lung, heart, brain, colon, rectum and skeletal muscle et al. were taken and fixed with 4% formalin. HE staining showed that the infiltrated tumor cells could often be found in spleen, lung, lymph node, and liver (FIG. 33). Immunohistochemistical analysis by using NOK antibody indicated that the infiltrated tumor cells were NOK positive and looked like lymphoid cells (FIG. 34). Subcutaneous injection of tumor cells prepared from the lymph nodes of NOK transgenic mice into nude mice resulted in tumor cell metastasis at multiple distant organs. The injected mice usually died within 2-3 weeks. Anatomical analysis showed the enlarged liver, spleen, and peripheral lymph nodes et al. that had been infiltrated by a large amount of tumor cells with typical lymphoid morphology. Immunohistochemistical analysis confirmed that these infiltrated tumor cells were NOK positive (FIG. 35).

### Example 20

### Development of B Cell Lymphoma/Leukemia Like Disease in NOK Transgenic Mice

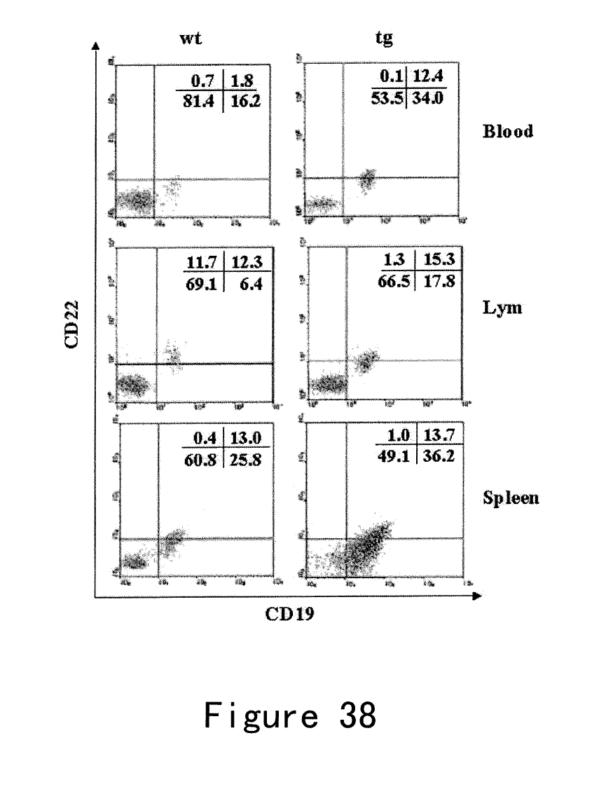

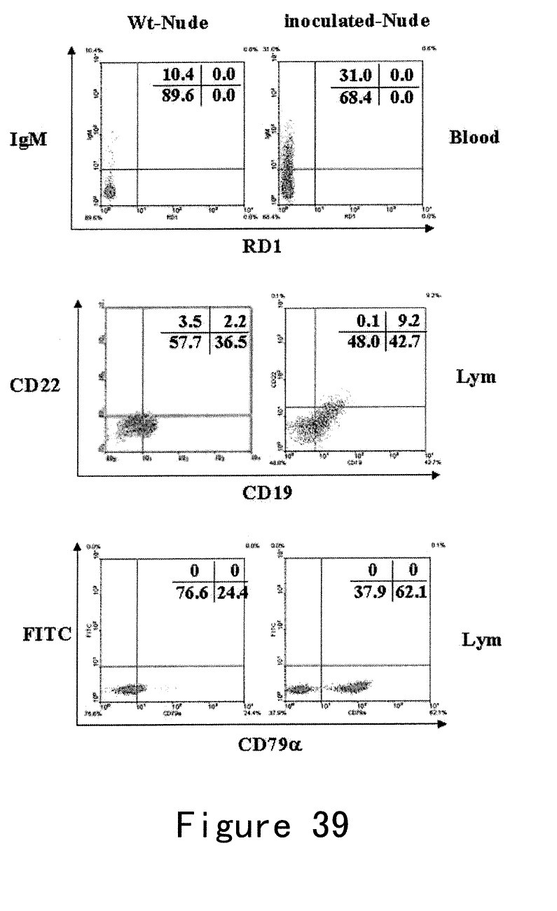

The results of blood routine test were often heterogeneous. The numbers of leukocytes in some transgenic mice were extremely high. However, the majority of them had a tendency of lower white blood cell counts with reduced numbers of mature lymphocytes and neutrophils and increased numbers of monocytes. In some instances, the monocytes could account for more than 50% of the whole leukocyte population. Leucocytes differential count of NOK transgenic mice was resemble to that of normal mice, but often with large amounts of degenerative lymphocytes and neutrophils. The enlarged lymph nodes usually developed at one side of the body and could reach to a diameter of more than 1-1.5 cm or even higher at late stage. Lymph node smear manifested a large amount of primitive and immature lymphocytes. Pox staining indicated that these tumor cells were indeed derived from lymphoid cells but not from granulocytes (FIG. 36). From a clinical point of view, these tumor cells were likely derived from B lymphocyte lineage. In order to define the tumor cell type, immune-typing on blood, spleen, and lymph node samples was conducted by flow cytometry analysis. An increased IgM⁺ population could be found in the blood, spleen, and lymph node of the NOK transgenic animal by using FITC-anti-mouse-IgM as a staining reagent, with a more dramatic increase found in lymph node sample (FIG. 37). The CD19⁺ B lymphocyte populations significantly increased in the peripheral blood, lymph node and spleen samples of NOK transgenic mice after double staining with FITC-anti-mouse CD22 and PE-anti-mouse CD19 (FIG. 38). Furthermore, the increased blood IgM⁺ B cells and increased CD19⁺/CD22⁺ and CD79α⁺ cell populations in lymph nodes were found in nude mice injected with cells prepared from the enlarged lymph nodes of NOK transgenic mice (FIG. 39). Thus, at least in some instances NOK-induced B cell lymphoma/leukemia should occur at a stage between the fractions of A, B/C of the pro-B cells and mature B cells during lymphocyte development according to Hardy's standard.

### Example 21

### Tumor Microarray Analysis Indicating Over-Expression of NOK in Different Types of Head and Neck Cancers and Other Solid Cancers

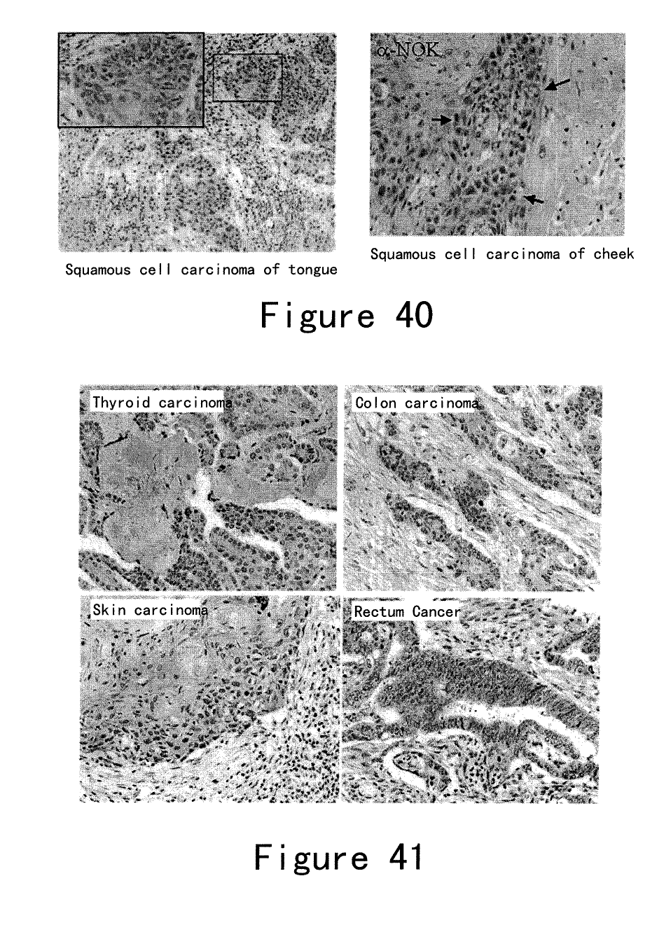

Tumor tissue microarray (TMA) was carried out in Cybrdi company (Xi'an, China) by using rabbit anti-NOK epitope antibody as described in example 13. TMA was conducted simultaneously on 50 individual samples collected from diverse head and neck cancers and 78 individual cancer samples isolated from different organs. The samples were incubated overnight with 1:1000 diluted rabbit anti-NOK antibody. Phosphate Buffer Saline (PBS) was used as mock control. Antigen retrieval was conducted by putting the section slides in 0.01M citric acid/sodium citrate buffer solution under high pressure and high temperature (95° C.) conditions. Immunohistochemistical staining was performed by using Streptavidin-Peroxidase kit (UltraSensitive™SP, Zhong Shan Company, Beijing). The endogenous peroxidase activity was blocked with 3% H₂O₂ in 1×PBS. Non-specific epitopes were blocked with un-immunized goat serum. The results demonstrated that NOK gene were up-regulated in a number of squamous cell carcinomas derived from tongue, cheek part and larynx (FIG. 40). Especially, NOK gene expression was reversely correlated with the pathogenetic grades of the squamous cell carcinoma of tongue. At high grade stage NOK gene expression was low, while at the lower grade stage NOK reached its high level of expression, indicating that the expression level of NOK gene was positively correlated with malignancy of this type of cancer. In addition, immunohistochemistical analysis indicated that NOK gene expression was also found to be up-regulated in numerous cancer types such as thyroid carcinoma, rectum ademocarcinoma, skin squamous cell type of carcinomas, colon ademocarcinoma and stomach mucous cell carcinoma et al., implying that NOK gene product might play a critical role in diverse cancer developments.

We claim:

1. An isolated polynucleotide comprising a nucleotide sequence selected from the group consisting of:
1) the nucleotide sequence of SEQ ID No: 1;
2) a nucleotide sequence encoding the amino acid sequence of SEQ ID No: 2; and
3) a nucleotide sequence having at least 90% sequence identity with that of 1) or 2),
Wherein the isolated polynucleotide encodes a product of Novel Oncogene with Kinase-domain (NOK).

2. The isolated polynucleotide according to claim 1, which encodes human NOK.

3. An isolated polynucleotide encoding a chimeric molecule fused between NOK and at least one heterogeneous polypeptide.

4. The isolated polynucleotide according to claim 3 comprising a nucleotide sequence selected from the group consisting of:
1) the nucleotide sequence of SEQ ID No: 5;
2) a nucleotide sequence encoding the amino acid sequence of SEQ ID No: 6; and
3) a nucleotide sequence having at least 90% sequence identity with that of 1) or 2).

5. The isolated polynucleotide according to claim 4, wherein the chimeric molecule is chimeric receptor EPOR/NOK which is a fusion between the extracellular domain of mouse EPOR and the transmembrane and intracellular domain of NOK.

6. The isolated polynucleotide according to anyone of claims 1 to 5, further comprising at its 3′ end a gen encoding a tag.

7. The isolated polynucleotide according to claim 6, wherein the tag is an HA tag or a FLAG tag.

8. The isolated polynucleotide according to claim 6, wherein the polynucleotide having the sequence of SEQ ID No: 3.

9. The isolated polynucleotide according to claim 6, wherein the polynucleotide having the sequence of SEQ ID No: 7.

10. An expression vector comprising a polynucleotide sequence according to anyone of claims 1 to 9.

11. An expression vector according to claim 10, which is the plasmid pcDNA3(NOK).

12. An expression vector according to claim 10, which is the plasmid pcDNA3(EPOR NOK).

13. A host cell transformed with an expression vector according to anyone of claims 10 to 12.

14. The host cell according to claim 13, which is BaF3-NOK cell line deposited in China General Microbiological Culture Collection Center (CGMCC) on May 9, 2004 with the deposit number of CGMCC No. 1145.

15. The host cell according to claim 13, which is BaF3-EPOR/NOK cell line deposited in China General Microbiological Culture Collection Center (CGMCC) on May 9, 2004 with the deposit number of CGMCC No. 1144.

16. An isolated polypeptide comprising the amino acid sequence of SEQ ID No: 2, or a biologically active fragment or derivative thereof.

17. The isolated polypeptide according to claim 16, wherein the derivative comprises an amino acid sequence derived from SEQ ID No: 2 by substitution, deletion or addition of one or several amino acid residues, and has the same biological activity as SEQ ID No: 2.

18. The isolated polypeptide according to claim 16 or 17, further comprising an HA tag at the carboxyl terminus.

19. The isolated polypeptide according to claim 18, wherein the polypeptide having the amino acid sequence of SEQ ID No: 4.

20. A fusion polypeptide, which is a chimeric molecule fused between NOK and at least one heterogeneous polypeptide.

21. A fusion polypeptide according to claim 20, which is a chimeric receptor EPOR/NOK fused between the extracellular domain of mouse EPOR and the transmembrane and intracellular domain of human NOK.

22. The fusion polypeptide according to claim 21, which has the amino acid sequence of SEQ ID No: 6, or an amino acid sequence derived from SEQ ID No: 6 by substitution, deletion or addition of one or several amino acid residues, and has the same biological activity as SEQ ID No: 6.

23. The fusion polypeptide according to anyone of claims 20 to 22, further comprising a FLAG tag at its carboxyl terminus.

24. The fusion polypeptide according to claim 23, wherein the fusion polypeptide having the amino acid sequence shown in SEQ ID No: 8.

25. A method for producing a polypeptide according to anyone of claims 16 to 24, comprising the steps of culturing a host cell according to anyone of claims 13 to 15 under conditions suitable for the expression of said polypeptide and collecting the expressed polypeptide.

26. A polypeptide epitope, wherein the polypeptide epitope has a sequence as shown in SEQ ID No: 10, corresponding to amino acid residues 360th to 380th of the amino acid sequence of NOK.

27. A polynucleotide encoding the polypeptide epitope according to claim 26, wherein the polynucleotide has a 61-nucleotide sequence as shown in SEQ ID No: 9, corresponding to the nucleotides from the 1078th to 1140th nucleotide of the conding sequence of human NOK.

28. An antibody specifically binding to a polypeptide according to anyone of claims 16 to 24.

29. The antibody according to claim 28 which specifically bind to the polypeptide epitope according to claim 26.

30. A mutant of EPOR/NOK comprising a single mutation at tyrosine residue 327 or 356 of NOK in the chimeric receptor EPOR/NOK.

31. An oligonucleotide probe or primer which is capable of hybridizing to a polynucleotide according to anyone of claims 1 to 9.

32. A transgenic animal comprising a polynucleotide according to anyone of claims 1 to 5.

33. A transgenic animal according to claim 32, which is mouse.

34. A method for screening an agent having anti-tumorigenesis and/or anti-metastasis activity, the method comprising determining the activity of a candidate agent to inhibit the proliferation of a cell according to anyone of claims 13 to 15, or to inhibit the tumorigenesis and metastasis in nude mice inoculated with a cell according to anyone of claims 13 to 15.

35. A method for detecting the presence of or the susceptibility to a disease in a subject, comprising detecting the presence or the mutation of the polynucleotide according to claim 1 or the polypeptide according to claim 16 in a biological sample from the subject.

36. The method according to claim 35, wherein the disease is selected from leukemia/lymphoma, head and neck cancer, thyroid carcinoma, gastrointestinal carcinoma and skin cancer.

37. A diagnostic kit comprising the antibody according to claim 28 or the oligonucleotide probe or primer according to claim 31.

38. A method for screening a therapeutic agent against tumorigenesis and/or metastasis comprising determining the activity of a candidate agent to inhibit tumorigenesis and/or metastasis in the transgenic mice according to claim 32 or 33.
